(19) United States
(12) Patent Application Publication
(10) Pub. No.: US 2018/0303603 A1
(43) Pub. Date: Oct. 25, 2018

(54) Medical Devices, Tissue Manipulators, Tissue Fixation Systems, and Associated Methods and Kits

(71) Applicants: Jeffry Melsheimer, Springville, IN (US); Rebecca Baumgartner, Bloomington, IN (US); Darin Schaeffer, Bloomington, IN (US); Nolan Xanh, West Lafayette, IN (US); Andrew Isch, Lafayette, IN (US); Shaun Tanner, West Lafayette, IN (US); Ali Ghamsari, West Lafayette, IN (US)

(72) Inventors: Jeffry Melsheimer, Springville, IN (US); Rebecca Baumgartner, Bloomington, IN (US); Darin Schaeffer, Bloomington, IN (US); Nolan Xanh, West Lafayette, IN (US); Andrew Isch, Lafayette, IN (US); Shaun Tanner, West Lafayette, IN (US); Ali Ghamsari, West Lafayette, IN (US)

(21) Appl. No.: 15/960,925

(22) Filed: Apr. 24, 2018

Related U.S. Application Data
(60) Provisional application No. 62/489,552, filed on Apr. 25, 2017.

(51) Int. Cl. A61F 2/20

(52) U.S. Cl. CPC A61F 2/20; A61F 2220/0008; A61F 2250/0036; A61F 2250/0037; A61F 2220/0041

(57) ABSTRACT

Tissue fixation systems and tissue manipulators are described herein. In addition, methods of using a tissue fixation system and a tissue manipulator, methods of manufacturing a tissue manipulator, and kits that include a tissue fixation system and a tissue manipulator are described herein. An example embodiment of a tissue fixation system includes a mounting plate, a mounting jaw releasably attached to the mounting plate, a mounting clamp releasably attached to the mounting plate, and a plurality of fasteners.

### RELATED APPLICATIONS

This application claims the benefit of U.S. Provisional Application No. 62/489,552, filed on Apr. 25, 2017. The disclosure of this related application is hereby incorporated into this disclosure by reference.

### FIELD

The disclosure relates generally to the field of medical devices. More particularly, the disclosure relates to medical devices, tissue manipulators, and tissue fixation systems. The disclosure also relates to methods that use, and kits that include, a tissue fixation system.

### BACKGROUND

Vocal cord paralysis may be associated with impaired breathing, swallowing, and phonation. Various procedures have been developed to reposition immobile vocal cords into a phonatory position to improve phonation, swallowing, and breathing. For example, some procedures require deep neck dissection to access the arytenoid and reposition the vocal cord via arytenoid adduction or arytenopexy coupled with cricothyroid approximation. However, these procedures have not gained wide acceptance because of the complexity of the dissection required. Another procedure, injection laryngoplasty, requires the injection of a material into the vocal folds. The results of injection laryngoplasty, however, can be temporary and sometimes require multiple injections to achieve the desired result. Another procedure, thyroplasty, requires the removal of a portion of the thyroid cartilage and the implantation of a piece of material that acts as a shim to push the vocal fold to the midline. This procedure is invasive and the results are usually not adjustable or reversible.

Therefore, a need exists for new and useful approaches to vocal cord treatment.

### SUMMARY OF SELECTED EXAMPLE EMBODIMENTS

Various medical devices, tissue manipulators, tissue fixation systems, methods, and kits are described herein.

An example tissue fixation system comprises a mounting plate, a mounting jaw, a mounting clamp, a plurality of fasteners, and a tissue manipulator. The mounting plate has a mounting plate front surface, a curved mounting plate back surface that opposably faces the mounting plate front surface, a mounting plate length that is measured along the mounting plate front surface, a mounting plate width that is measured along the mounting plate front surface and orthogonal to the mounting plate length, a mounting plate thickness that extends from the mounting plate front surface to the mounting plate back surface, and a mounting plate main body. The mounting plate main body defines a mounting plate passageway that extends through the mounting plate thickness, a mounting plate track that extends along a portion of the mounting plate width, and first, second, and third mounting plate apertures that extend through the mounting plate thickness. The mounting jaw is releasably attached to the mounting plate and partially covers the mounting plate passageway. The mounting jaw has a mounting jaw main body that defines a mounting jaw aperture. The mounting clamp is releasably attached to the mounting plate and is partially disposed over the mounting plate track. The mounting clamp has a mounting clamp main body that defines a mounting clamp first aperture and a mounting plate second aperture. A first attachment member of the plurality of attachment members is partially disposed through the mounting plate first aperture and the mounting jaw aperture and provides releasable attachment between the mounting plate and mounting jaw. A second attachment member of the plurality of attachment members is partially disposed through the mounting plate second aperture and the mounting clamp first aperture and provides releasable attachment between the mounting plate and mounting clamp. A third attachment member of the plurality of attachment members is partially disposed through the mounting plate third aperture and the mounting clamp second aperture and provides releasable attachment between the mounting plate and mounting clamp. The tissue manipulator has a shaft and a coil. The shaft is partially disposed within the mounting plate track and between the mounting plate and the mounting clamp. The shaft is partially disposed between the mounting plate and the mounting jaw.

Another example tissue fixation system comprises a mounting plate, a mounting jaw, a mounting clamp, a plurality of fasteners, and a tissue manipulator. The mounting plate has a mounting plate front surface, a curved mounting plate back surface that is opposably faces the mounting plate front surface, a mounting plate length that is measured along the mounting plate front surface, a mounting plate width that is measured along the mounting plate front surface and orthogonal to the mounting plate length, a mounting plate thickness that extends from the mounting plate front surface to the mounting plate back surface, and a mounting plate main body. The mounting plate main body defines a mounting plate recess that extends from the mounting plate front surface toward the mounting plate back surface to a mounting plate recess base, a mounting plate passageway that extends from the mounting plate recess base to the mounting plate back surface, a mounting plate track that is disposed on the mounting plate recess base that extends along a portion of the mounting plate width, and first, second, third, fourth, and fifth mounting plate apertures that extend through the mounting plate thickness. Each of the mounting plate first aperture, the mounting plate second aperture, and the mounting plate third aperture extends from the mounting plate recess base to the mounting plate back surface. The mounting plate track comprises a mounting plate track projection that extends from the mounting plate recess base and away from the mounting plate back surface. The mounting jaw is releasably attached to the mounting plate and partially covers the mounting plate passageway. The mounting jaw is disposed within the mounting plate recess and has a mounting jaw main body that defines a mounting jaw aperture. The mounting clamp is releasably attached to the mounting plate and is partially disposed over the mounting plate track. The mounting clamp is disposed within the mounting plate recess and has a mounting clamp main body that defines a mounting clamp first aperture and a mounting plate second aperture. A first attachment member of the plurality of attachment members is partially disposed through the mounting plate first aperture and the mounting jaw aperture and provides releasable attachment between the mounting plate and mounting jaw. A second attachment member of the plurality of attachment members is partially disposed through the mounting plate second aperture and the mounting clamp first aperture and provides releasable attachment between the mounting plate and mounting clamp. A third attachment member of the plurality of attachment members is partially disposed through the mounting plate third aperture and the mounting clamp second aperture and provides releasable attachment between the mounting plate and mounting clamp. The tissue manipulator has a shaft and a coil. The shaft is partially disposed within the mounting plate track and between the mounting plate and the mounting clamp. The shaft is partially disposed between the mounting plate and the mounting jaw.

Another example tissue fixation system comprises a mounting plate, a mounting jaw, a mounting clamp, a plurality of fasteners, and a tissue manipulator. The mounting plate has a mounting plate front surface, a curved mounting plate back surface that opposably faces the mounting plate front surface, a mounting plate length that is measured along the mounting plate front surface, a mounting plate width that is measured along the mounting plate front surface and orthogonal to the mounting plate length, a mounting plate thickness that extends from the mounting plate front surface to the mounting plate back surface, and a mounting plate main body. The mounting plate main body defines a mounting plate recess that extends from the mounting plate front surface toward the mounting plate back surface to a mounting plate recess base, a mounting plate passageway that extends from the mounting plate recess base to the mounting plate back surface, a mounting plate track disposed on the mounting plate recess base that extends along a portion of the mounting plate width, and first, second, third, fourth, and fifth mounting plate apertures that extend through the mounting plate thickness. Each of the mounting plate first aperture, mounting plate second aperture, and mounting plate third aperture extends from the mounting plate recess base to the mounting plate back surface. The mounting plate track comprises a mounting plate track projection and a mounting plate track recess. The mounting plate projection extends from the mounting plate recess base and away from the mounting plate back surface. The mounting plate track recess extends into the mounting plate track and toward the mounting plate back surface. The mounting jaw is releasably attached to the mounting plate and partially covers the mounting plate passageway. The mounting jaw is disposed within the mounting plate recess and has a mounting jaw main body that defines a mounting jaw aperture. The mounting clamp is releasably attached to the mounting plate and is partially disposed over the mounting plate track. The mounting clamp is disposed within the mounting plate recess and has a mounting clamp main body that defines a mounting clamp first aperture and a mounting plate second aperture. A first attachment member of the plurality of attachment members is partially disposed through the mounting plate first aperture and the mounting jaw aperture and provides releasable attachment between the mounting plate and mounting jaw. A second attachment member of the plurality of attachment members is partially disposed through the mounting plate second aperture and the mounting clamp first aperture and provides releasable attachment between the mounting plate and mounting clamp. A third attachment member of the plurality of attachment members is partially disposed through the mounting plate third aperture and the mounting clamp second aperture and provides releasable attachment between the mounting plate and mounting clamp. A portion of the mounting plate first aperture and a portion of the mounting plate second aperture are disposed on a first hypothetical plane. A portion of the mounting plate first aperture and a portion of the mounting plate third aperture are disposed on a second hypothetical plane. A portion of the mounting plate first aperture, a portion of the mounting plate passageway, and a portion of the mounting plate track are disposed on a third hypothetical plane that is disposed between the first hypothetical plane and the second hypothetical plane. The third hypothetical plane is disposed at an angle to the first hypothetical plane that is less than 90 degrees. The third hypothetical plane is disposed at an angle to the second hypothetical plane that is less than 90 degrees. The tissue manipulator has a shaft and a coil. The shaft is partially disposed within the mounting plate track and between the mounting plate and the mounting clamp. The shaft is partially disposed between the mounting plate and the mounting jaw.

An example medical device comprises a mounting plate, a mounting jaw, a mounting clamp, and a plurality of fasteners. The mounting plate has a mounting plate front surface, a curved mounting plate back surface that opposably faces the mounting plate front surface, a mounting plate length that is measured along the mounting plate front surface, a mounting plate width that is measured along the mounting plate front surface and orthogonal to the mounting plate length, a mounting plate thickness that extends from the mounting plate front surface to the mounting plate back surface, and a mounting plate main body. The mounting plate main body defines a mounting plate passageway that extends through the mounting plate thickness, a mounting plate track that extends along a portion of the mounting plate width, and first, second, and third mounting plate apertures that extend through the mounting plate thickness. The mounting jaw is releasably attached to the mounting plate and partially covers the mounting plate passageway. The mounting jaw has a mounting jaw main body that defines a mounting jaw aperture. The mounting clamp is releasably attached to the mounting plate and is partially disposed over the mounting plate track. The mounting clamp has a mounting clamp main body that defines a mounting clamp first aperture and a mounting plate second aperture. A first attachment member of the plurality of attachment members is partially disposed through the mounting plate first aperture and the mounting jaw aperture and provides releasable attachment between the mounting plate and mounting jaw. A second attachment member of the plurality of attachment members is partially disposed through the mounting plate second aperture and the mounting clamp first aperture and provides releasable attachment between the mounting plate and mounting clamp. A third attachment member of the plurality of attachment members is partially disposed through the mounting plate third aperture and the mounting clamp second aperture and provides releasable attachment between the mounting plate and mounting clamp.

An example tissue manipulator has a tissue manipulator first end, a tissue manipulator second end, a tissue manipulator length, a shaft, and a coil. The tissue manipulator length extends from the tissue manipulator first end to the tissue manipulator second end. The shaft has a shaft lengthwise axis, a shaft first end, a shaft second end, and a shaft main body that has a predefined shaft bend. The coil is disposed between the tissue manipulator first end and the tissue manipulator second end and is attached to the shaft at the shaft second end.

An example method of manufacturing a tissue manipulator comprises the steps of: selecting a wire member to form a tissue manipulator; reducing the outside diameter of the wire member along a portion of its length; forming one or more turns along a portion of the length of the wire member to form a coil; trimming the wire member to a desired length; forming a sharp tip on an end of the wire member; forming a bend on the wire member.

An example method of treatment using a tissue fixation system, such as a method of repositioning an arytenoid muscle which results in the repositioning of a vocal cord, comprises the steps of: creating an opening in the body over the vocal cord intended to be treated to access the thyroid cartilage; introducing a needle and an obturator through the thyroid cartilage and into the arytenoid muscle; withdrawing the obturator while maintaining position of needle; passing a tissue manipulator through the needle until it contacts the arytenoid muscle; applying torque to the tissue manipulator while applying an axial force toward the arytenoid muscle such that the tissue manipulator rotates and engages the arytenoid muscle; trimming the tissue manipulator between the shaft bend and the shaft second end to create a new tissue manipulator end; withdrawing the needle from the tissue and thyroid cartilage while maintaining position of the tissue manipulator; applying an axial force on the tissue manipulator to achieve a desired placement of vocal cord; marking the shaft of the tissue manipulator where it exits the thyroid cartilage while holding the tissue manipulator at desired location; passing the new tissue manipulator end through the mounting plate passageway of a mounting plate; attaching the mounting plate to the thyroid cartilage; positioning the marking indicia on the tissue manipulator adjacent to the mounting plate passageway; attaching the mounting jaw to the mounting plate such that a portion of the tissue manipulator is disposed between the mounting plate and the mounting jaw; bending the portion of the tissue manipulator extending from the mounting plate at the marking indicia on the tissue manipulator toward the midline of the throat; attaching the mounting clamp to the mounting plate such that a portion of the tissue manipulator is disposed between the mounting plate and the mounting clamp; trimming tissue manipulator; asking the patient to phonate to evaluate the position of the vocal cord and to determine whether adjustments are needed; and closing the opening made in the body.

Additional understanding of the exemplary medical devices, tissue manipulators, tissue fixation systems, methods that use a tissue fixation system, and kits that include a tissue fixation system can be obtained by review of the detailed description, below, and the appended drawings.

### BRIEF DESCRIPTION OF THE DRAWINGS

FIG. 1 is a perspective view of a first example medical device.

FIG. 2 is an exploded view of the medical device illustrated in FIG. 1.

FIG. 3 is a perspective view of the mounting plate of the medical device illustrated in FIG. 1.

FIG. 4 is a perspective view of the mounting jaw of the medical device illustrated in FIG. 1.

FIG. 5 is a perspective view of the mounting clamp of the medical device illustrated in FIG. 1.

FIG. 6 is a perspective view of a second example medical device.

FIG. 7 is an exploded view of the medical device illustrated in FIG. 6.

FIG. 8 is a top view of the mounting plate of the medical device illustrated in FIG. 6.

FIG. 9 is an elevation view of the mounting plate illustrated in FIG. 8.

FIG. 10 is a perspective view of the mounting plate illustrated in FIG. 8.

FIG. 11 is a perspective view of the mounting jaw of the medical device illustrated in FIG. 6.

FIG. 12 is an elevation view of the mounting jaw illustrated in FIG. 11.

FIG. 13 is a perspective view of the mounting clamp of the medical device illustrated in FIG. 6.

FIG. 14 is another perspective view of the mounting clamp illustrated in FIG. 13.

FIG. 15 is an elevation view of the mounting clamp illustrated in FIG. 13.

FIG. 16 is a perspective view of an alternative mounting plate that can be included in a medical device.

FIG. 17 is a perspective view of a third example medical device.

FIG. 18 is an elevation view of the medical device illustrated in FIG. 17.

FIG. 19 is an exploded view of the medical device illustrated in FIG. 17.

FIG. 20 is a top view of the mounting plate of the medical device illustrated in FIG. 17.

FIG. 21 is a perspective view of the mounting plate illustrated in FIG. 20.

FIG. 22 is an elevation view of the mounting plate illustrated in FIG. 20.

FIG. 23 is a side view of the mounting plate illustrated in FIG. 20.

FIG. 24 is a perspective view of a fourth example medical device.

FIG. 25 is another perspective view of the medical device illustrated in FIG. 24.

FIG. 26 is an elevation view of the medical device illustrated in FIG. 24.

FIG. 27 is a perspective view of the mounting plate of the medical device illustrated in FIG. 24.

FIG. 28 is another perspective view of the mounting plate illustrated in FIG. 27.

FIG. 29 is a perspective view of the mounting jaw of the medical device illustrated in FIG. 24.

FIG. 30 is an elevation view of the mounting jaw illustrated in FIG. 29.

FIG. 31 is a perspective view of the mounting clamp of the medical device illustrated in FIG. 24.

FIG. 32 is another perspective view of the mounting clamp illustrated in FIG. 31.

FIG. 33 is a side view of the mounting clamp illustrated in FIG. 31.

FIG. 34 is a perspective view, partially broken away, of a first example tissue manipulator.

FIG. 35 is a magnified view of area I in FIG. 34.

FIG. 35A is a perspective view, partially broken away, of a second example tissue manipulator.

FIG. 36 is a perspective view, partially broken away, of a third example tissue manipulator.

FIG. 37 is a perspective view, partially broken away, of a fourth example tissue manipulator.

FIG. 38 is a schematic illustration of an example method of manufacturing a tissue manipulator.

FIG. 39 illustrates an example kit that includes a tissue fixation system.

FIG. 40 is a schematic illustration of an example method of treatment using a tissue fixation system.

FIG. 41 illustrates a superior view of the thyroid cartilage and the arytenoid muscle with a needle and obturator disposed through the thyroid cartilage and partially disposed within the arytenoid muscle.

FIG. 42 illustrates a superior view of the thyroid cartilage and the arytenoid muscle with a needle disposed through the thyroid cartilage and partially disposed within the arytenoid muscle.

FIG. 43 illustrates a superior view of the thyroid cartilage and the arytenoid muscle with a needle and a tissue manipulator disposed through the thyroid cartilage and partially disposed within the arytenoid muscle.

FIG. 44 illustrates a superior view of the thyroid cartilage and the arytenoid muscle with a needle and a tissue manipulator disposed through the thyroid cartilage and partially disposed within the arytenoid muscle. The tissue manipulator has been advanced into the arytenoid muscle.

FIG. 45 illustrates a superior view of the thyroid cartilage and the arytenoid muscle with a tissue manipulator disposed through the thyroid cartilage and partially disposed within the arytenoid muscle.

FIG. 46 illustrates a superior view of the thyroid cartilage and the arytenoid muscle with a tissue manipulator disposed through the thyroid cartilage and partially disposed within the arytenoid muscle. The tissue manipulator has repositioned the arytenoid muscle and includes a marking indicia along its length adjacent the thyroid cartilage.

FIG. 47 illustrates a superior view of the thyroid cartilage and the arytenoid muscle with a mounting plate of a medical device attached to the thyroid cartilage and a tissue manipulator disposed through the mounting plate passageway and partially disposed within the arytenoid muscle.

FIG. 48 illustrates a superior view of the thyroid cartilage and the arytenoid muscle with a mounting plate and a mounting jaw attached to the thyroid cartilage and a tissue manipulator disposed through the mounting plate passageway and partially disposed within the arytenoid muscle.

FIG. 49 illustrates a superior view of the thyroid cartilage and the arytenoid muscle with a mounting plate, a mounting jaw, and a mounting clamp attached to the thyroid cartilage and a tissue manipulator disposed through the mounting plate passageway and partially disposed within the arytenoid muscle.

FIG. 50 is a cross-sectional view of the medical device illustrated in FIG. 1 attached to the thyroid cartilage and a tissue manipulator disposed through the mounting plate passageway and the thyroid cartilage.

FIG. 51 is a cross-sectional view of the medical device illustrated in FIG. 6 attached to the thyroid cartilage and a tissue manipulator disposed through the mounting plate passageway and the thyroid cartilage.

FIG. 52 is a cross-sectional view of the medical device illustrated in FIG. 17 attached to the thyroid cartilage and a tissue manipulator disposed through the mounting plate passageway and the thyroid cartilage.

FIG. 53 is a cross-sectional view of the medical device illustrated in FIG. 24 attached to the thyroid cartilage and a tissue manipulator disposed through the mounting plate passageway and the thyroid cartilage.

### DETAILED DESCRIPTION

The following detailed description and the appended drawings describe and illustrate various example embodiments of medical devices, tissue manipulators, tissue fixation systems, and associated methods and kits. The description and illustration of these examples are provided to enable one skilled in the art to make and use a medical device, a tissue manipulator, a tissue fixation system, to practice a method of manufacturing a tissue manipulator, to practice a method of treatment using a tissue fixation system, and to make a kit that includes a tissue fixation system. They are not intended to limit the scope of the claims in any manner.

As used herein, the term “engaged” and grammatically related terms refer to a first element or feature being associated with, interconnected with, disposed in, or attached to a second element or feature such that the first element or feature can alter the position of the second element or feature when an outside force is applied on a portion of the first element or feature.

As used herein, the term “fixation” refers to the ability of a first device, element, or feature to maintain a second device, element, or feature in a particular position.

As used herein, the phrase “arytenoid muscle” refers to the arytenoid muscle, the thyroarytenoid muscle, the cricoarytenoid muscle, and the arytenoid muscular process.

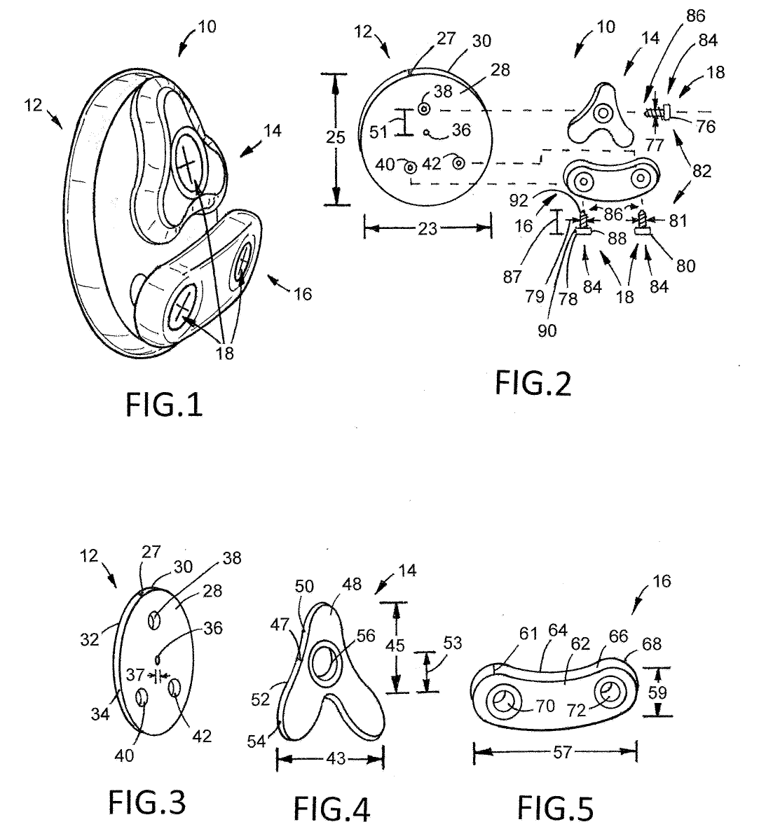

FIGS. 1, 2, 3, 4, and 5 illustrate a first example medical device 10. The medical device 10 includes a mounting plate 12, a mounting jaw 14, a mounting clamp 16, and a plurality of fasteners 18. In the illustrated embodiment, the mounting jaw 14 is releasably attached to the mounting plate 12 using a first fastener 76 and the mounting clamp 16 is releasably attached to the mounting plate 12 using a second fastener 78 and a third fastener 80, as described in more detail herein. Although not illustrated in FIGS. 1, 2, 3, 4, and 5, a medical device can alternatively include fourth and fifth fasteners to assist with attachment of the medical device to a portion of the body (e.g., thyroid cartilage). When included in a medical device, such as medical device 10, each of the fourth and fifth fasteners can be passed through a mounting plate main body and into the portion of the body to which it is intended to attach a medical device prior to, during, or subsequent to, attachment of a mounting jaw and/or mounting clamp to the mounting plate.

In the illustrated embodiment, the mounting plate 12 has a mounting plate length 23, a mounting plate width 25, a mounting plate thickness 27, a mounting plate front surface 28, a mounting plate back surface 30, a mounting surface side 32, and a mounting plate main body 34 that defines a mounting plate passageway 36, a mounting plate first aperture 38, a mounting plate second aperture 40, and a mounting plate third aperture 42. The mounting plate length 23 is measured along the mounting plate front surface 28. The mounting plate width 25 is measured along the mounting plate front surface 28 and orthogonal to the mounting plate length 23. The mounting plate thickness 27 is measured along the mounting plate side 32 and extends from the mounting plate front surface 28 to the mounting plate back surface 30. In the illustrated embodiment, the mounting plate thickness 27 is constant along the mounting plate length 23 and mounting plate width 25. However, as described herein, alternative embodiments can include a mounting plate that has a mounting plate thickness that varies along the mounting plate length and/or mounting plate width to accommodate the thickness of a mounting jaw and/or mounting clamp.

In the illustrated embodiment, the mounting plate length 23 is equal to the mounting plate width 25 such that the mounting plate 12 is cylindrical with a curved mounting plate side 32. While the mounting plate 12 has been illustrated as being cylindrical with a curved mounting plate side 32, a mounting plate can have any suitable structural configuration and selection of a suitable structural configuration can be based on various considerations, such as the anatomy of the patient and/or the structural configuration of a mounting jaw and/or mounting clamp. Example structural configurations considered suitable for a mounting plate include mounting plates that are cylindrical, elongated cylinders, prisms, elongated prisms, octagonal prisms, triangular prisms, ovoids, cuboids, variations of those described herein, and any other structural configuration considered suitable for a particular embodiment.

In the illustrated embodiment, the mounting plate front surface 28 is substantially flat (e.g., a majority, or entity, of the surface is disposed on a plane) and the mounting plate back surface 30 is opposably facing the mounting plate front surface 28 (e.g., a majority, or entity, of the back surface faces a direction that is opposite the direction that a majority, or entity, of front surface faces) and is substantially flat (e.g., a majority, or entity, of the surface is disposed on a plane). While each of the mounting plate front surface 28 and mounting plate back surface 30 has been illustrated as having a particular structural configuration, the mounting plate front surface and mounting plate back surface of a mounting plate can have any suitable configuration and selection of a suitable configuration for the surface of a mounting plate can be based on various considerations, such as the anatomy of the patient being treated. Examples of other configurations considered suitable for the mounting plate front surface and/or mounting plate back surface of a mounting plate include surfaces that define a pre-defined curve across the entirety, or a portion, of the mounting plate length and/or mounting plate width, a partial curve, surfaces that are substantially flat, and any other configuration considered suitable for a particular embodiment. For example, the mounting plate front surface and/or mounting plate back surface of a mounting plate can include a pre-formed curve that extends along the entirety, or a portion, of the mounting plate length and/or mounting plate width to assist with attachment to the thyroid cartilage of a patient.

The mounting plate side 32 extends from the mounting plate front surface 28 to the mounting plate back surface 30 and defines a continuous curve from the mounting plate front surface 28 to the mounting plate back surface 30. This structural configuration provides a mechanism for reducing trauma to tissue during, and subsequent to, implantation of the medical device 10 in a patient. While the mounting plate side 32 has been illustrated as defining a continuous curve, the mounting plate side of a mounting plate can have any suitable configuration and selection of a suitable configuration for a mounting plate side can be based on various considerations, such as the anatomy of the patient being treated. Examples of other configurations considered suitable for the mounting plate side of a mounting plate include curved, partially curved, multi-faceted, and any other configuration considered suitable for a particular embodiment.

The mounting plate passageway 36 extends from the mounting plate front surface 28 to the mounting plate back surface 30 and is sized and configured to receive a medical device, such as a portion of a tissue manipulator, as described in more detail herein. In the illustrated embodiment, the mounting plate passageway 36 has an inside diameter 37 and is disposed between a first hypothetical plane that contains a portion of the mounting plate first aperture 38 and a portion of the mounting plate second aperture 40, a second hypothetical plane that contains a portion of the mounting plate first aperture 38 and a portion of the mounting plate third aperture 42, and a third hypothetical plane that contains a portion of the mounting plate second aperture 40 and a portion of the mounting plate third aperture 42. While the mounting plate passageway 36 has been illustrated as positioned at a particular location on the mounting plate 12, a mounting plate passageway can be positioned at any suitable location on a mounting plate and selection of a suitable location can be based on various considerations, such as the structural configuration of a mounting jaw and/or mounting clamp included in a medical device. Examples of suitable locations to position a mounting plate passageway on a mounting plate include in a central location (e.g., in the center of the mounting plate), adjacent the mounting plate first aperture, the mounting plate second aperture, and/or the mounting plate third aperture, and any other location considered suitable for a particular embodiment. In addition, the inside diameter of a mounting plate passageway can be greater than, equal to, substantially equal to, or less than the outside diameter of a portion of a tissue manipulator (e.g., shaft) intended to be passed through the mounting plate passageway.

Each of the mounting plate first aperture 38, mounting plate second aperture 40, and mounting plate third aperture 42 extends from the mounting plate front surface 28 to the mounting plate back surface 30 and is sized and configured to receive a fastener of the plurality of fasteners 18. As described above, each of the first, second, and mounting plate third apertures 38, 40, 42 has been illustrated as positioned at a particular location on the mounting plate main body 34. However, a mounting plate aperture can be positioned at any suitable location on a mounting plate and selection of a suitable location can be based on various considerations, such as the structural configuration of a mounting jaw and/or mounting clamp included in a medical device. For example, a portion of a mounting plate first aperture can be positioned on a first hypothetical plane that is parallel to a second hypothetical plane that contains the center of a mounting plate and/or a portion of the mounting plate passageway such that the mounting plate first aperture is offset from mounting plate passageway relative to the center of the mounting plate. Alternatively, or in combination with the variations described herein, a portion of a mounting plate second aperture and/or a mounting plate third aperture can be disposed on a first hypothetical plane that is disposed orthogonally to a second hypothetical plane that contains a portion of the mounting plate first aperture and/or a portion of a mounting plate passageway.

While the mounting plate 12 has been illustrated as including a first, second, and third mounting plate apertures 38, 40, 42, the main body of a mounting plate can define any suitable number of apertures and selection of a suitable number of apertures to define on a mounting plate can be based on various considerations, such as the configuration of a mounting jaw and/or mounting clamp included in a medical device. Examples of numbers of apertures considered suitable to define on a mounting plate include one, at least one, two, a plurality, three, four, five, six, seven, and any other number considered suitable for a particular embodiment. The number of apertures defined by a mounting plate can be based on the number of apertures defined by a mounting jaw and/or mounting clamp included in a medical device and/or the number of apertures intended to assist with attachment of the mounting plate to the wall of a body member (e.g., thyroid cartilage). For example, an alternative embodiment of a mounting plate can include a mounting plate fourth aperture and a mounting plate fifth aperture that each extends from the mounting plate front surface to the mounting plate back surface and is sized and configured to receive a fastener of a plurality of fasteners. In alternative embodiments that include a mounting plate fourth aperture, the mounting plate fourth aperture can be disposed between the mounting plate side of a mounting plate and a mounting plate passageway and partially disposed between the mounting plate front surface and the mounting jaw. In alternative embodiments that include a mounting plate fifth aperture, the mounting plate fifth aperture can be disposed between the mounting plate side of a mounting plate and a mounting plate passageway and partially disposed between the mounting plate front surface and the mounting jaw.

In the illustrated embodiment, the mounting jaw 14 is a substantially triangular prism and has a mounting jaw length 43, a mounting jaw width 45, a mounting jaw thickness 47, a mounting jaw front surface 48, a mounting jaw back surface 50, a mounting jaw side 52, and a mounting jaw main body 54 that defines a mounting jaw aperture 56. The mounting jaw length 43 is measured along the mounting jaw front surface 48 and is greater than the inside diameter 37 of the mounting plate passageway 36. The mounting jaw width 45 is measured along the mounting plate front surface 48 and orthogonal to the mounting plate length 43. The mounting jaw thickness 47 is measured along the mounting jaw side 52 and extends from the mounting jaw front surface 48 to the mounting jaw back surface 50.

In the illustrated embodiment, the mounting jaw length 43 is less than the mounting plate length 23 and greater than the mounting jaw width 45. While the mounting jaw 14 has been illustrated as being a substantially triangular prism, a mounting jaw can have any suitable structural configuration and selection of a suitable structural configuration can be based on various considerations, such as the anatomy of the patient and the structural configuration of a mounting plate and/or mounting clamp. Example structural configurations considered suitable for a mounting jaw include mounting jaws that are cylindrical, elongated cylinders, prisms, elongated prisms, octagonal prisms, triangular prisms, ovoids, cuboids, variations of those described herein, and any other structural configuration considered suitable for a particular embodiment.

In the illustrated embodiment, the mounting jaw front surface 48 is curved along the entirety of the mounting jaw length 43 and mounting jaw width 45 and the mounting jaw back surface 50 is substantially flat (e.g., a majority, or entity, of the surface is disposed on a plane) and opposably facing the mounting jaw front surface 48 (e.g., a majority, or entity, of the back surface faces a direction that is opposite the direction that a majority, or entity, of front surface faces). The curvature of the mounting jaw front surface 48 provides a mechanism for reducing trauma to tissue during, and subsequent to, implantation of the medical device 10 in a patient. While the mounting jaw front surface 48 has been illustrated as curved and the mounting jaw back surface 50 has been illustrated as substantially flat, the mounting jaw front surface and mounting jaw back surface of a mounting jaw can have any suitable configuration and selection of a suitable configuration for a surface of a mounting jaw can be based on various considerations, such as the anatomy of the patient being treated. Examples of other configurations considered suitable for the mounting jaw front surface and/or mounting jaw back surface of a mounting jaw include surfaces that define a pre-defined curve across the entirety, or a portion, of the mounting jaw length and/or mounting jaw width, a partial curve, surfaces that are substantially flat, and any other configuration considered suitable for a particular embodiment. For example, a mounting jaw can include a mounting jaw back surface that is sized and configured to mate with, or mirrors, a portion of the mounting plate front surface of a mounting plate.

The mounting jaw side 52 extends from the mounting jaw front surface 48 to the mounting jaw back surface 50 and defines a continuous curve from the mounting jaw front surface 48 to the mounting jaw back surface 50. This structural configuration provides a mechanism for reducing trauma to tissue during, and subsequent to, implantation of the medical device 10 in a patient. While the mounting jaw side 52 has been illustrated as defining a continuous curve, the mounting jaw side of a mounting jaw can have any suitable configuration and selection of a suitable configuration for a mounting jaw side can be based on various considerations, such as the anatomy of the patient being treated. Examples of other configurations considered suitable for the mounting jaw side of a mounting jaw include curved, partially curved, multi-faceted, and any other configuration considered suitable for a particular embodiment.

The mounting jaw aperture 56 extends from the mounting jaw front surface 48 to the mounting jaw back surface 50 and is sized and configured to receive a fastener of the plurality of fasteners 18 (e.g., has a diameter that is greater than the major diameter of a fastener intended to be passed through the aperture). In the illustrated embodiment, a first distance 51 extends from the center of the mounting plate first aperture 38 to the center of the mounting plate passageway 36 and a second distance 53 extends from the center of the mounting jaw aperture 56 to the mounting jaw side 52. Each of the first distance and 51 and second distance 53 is measured along a plane that contains the center of the mounting plate first aperture 38 and the center of the mounting plate passageway 36. In the illustrated embodiment, the first distance 51 is less than the second distance 53 such that a portion of the mounting jaw 14 is disposed over the mounting plate passageway 36 when the medical device 10 is assembled.

In the illustrated embodiment, the mounting clamp 16 has mounting clamp length 57, a mounting clamp width 59, a mounting clamp thickness 61, a mounting clamp front surface 62, a mounting clamp back surface 64, a mounting clamp side 66, and a mounting clamp main body 68 that defines a mounting clamp first aperture 70 and a mounting clamp second aperture 72. The mounting clamp length 57 is measured along the mounting clamp front surface 62 and is less than the mounting plate length 23, greater than the mounting jaw length 43, and greater than the mounting clamp width 59. The mounting clamp width 59 is measured along the mounting clamp front surface 62 and orthogonal to the mounting clamp length 57. The mounting clamp thickness 61 is measured along the mounting clamp side 66 and extends from the mounting jaw front surface 62 to the mounting jaw back surface 64.

In the illustrated embodiment, the mounting clamp 16 is a substantially elongated cuboid. While the mounting clamp 16 has been illustrated as being a substantially elongated cuboid and having a mounting clamp length 57 that is less than the mounting plate length 23, greater than the mounting jaw length 43, and the mounting clamp width 59, a mounting clamp can have any suitable structural configuration and selection of a suitable structural configuration can be based on various considerations, such as the anatomy of the patient and the structural configuration of a mounting plate and/or mounting jaw. Examples of structural configurations considered suitable for a mounting clamp include mounting clamps that are cylindrical, elongated cylinders, prisms, elongated prisms, octagonal prisms, triangular prisms, ovoids, cuboids, mounting clamps that have a length less than, equal to, substantially equal to, or greater than the mounting plate length of a mounting plate, the mounting jaw length of a mounting jaw, and/or the mounting clamp length of a mounting clamp, variations of those described herein, and any other structural configuration considered suitable for a particular embodiment.

In the illustrated embodiment, each of the mounting clamp front surface 62 and the mounting clamp back surface 64 is curved along the entirety of the mounting clamp length 57 and the mounting clamp back surface 64 is opposably facing the mounting clamp front surface 62. The curvature of the mounting clamp front surface 62 and mounting plate back surface 66 provides a mechanism for mounting the mounting clamp over a tissue manipulator, such as those described herein, and reducing trauma to tissue during, and subsequent to, implantation of the medical device 10 in a patient. While the mounting clamp front surface 62 and mounting clamp back surface 64 have been illustrated as curved, the mounting clamp front surface and mounting clamp back surface of a mounting clamp can have any suitable configuration and selection of a suitable configuration for a mounting clamp front surface and/or mounting clamp back surface can be based on various considerations, such as the anatomy of the patient being treated. Examples of other configurations considered suitable for the mounting clamp front surface and/or mounting clamp back surface of a mounting clamp include surfaces that define a pre-defined curve across the entirety, or a portion, of the mounting clamp length and/or mounting jaw clamp, a partial curve, surfaces that are substantially flat, and any other configuration considered suitable for a particular embodiment. For example, a mounting clamp can include a mounting clamp back surface that is sized and configured to mate with, or mirrors, a portion of the mounting plate front surface of a mounting plate.

The mounting clamp side 66 extends from the mounting clamp front surface 62 to the mounting clamp back surface 64 and defines a continuous curve from the mounting clamp front surface 62 to the mounting clamp back surface 64. This structural configuration provides a mechanism for reducing trauma to tissue during, and subsequent to, implantation of the medical device 10 in a patient. While the mounting clamp side 66 has been illustrated as defining a continuous curve, the mounting clamp side of a mounting clamp can have any suitable configuration and selection of a suitable configuration for a mounting clamp side can be based on various considerations, such as the anatomy of the patient being treated. Examples of other configurations considered suitable for the mounting clamp side of a mounting clamp include curved, partially curved, multi-faceted, and any other configuration considered suitable for a particular embodiment.

Each of the mounting clamp first aperture 70 and mounting clamp second aperture 72 extends from the mounting clamp front surface 62 to the mounting clamp back surface 64 and is sized and configured to receive a fastener of the plurality of fasteners 18 (e.g., has a diameter that is greater than the major diameter of a fastener intended to be passed through the aperture). In the illustrated embodiment, each aperture 38, 40, 42, 56, 70, 72 defined by a mounting plate, mounting jaw, and mounting clamp is countersunk such that it is sized and configured to receive a portion of a fastener head, as described in more detail herein. However, alternative embodiment can include apertures that are not countersunk and/or passageways that are countersunk. Optionally, each aperture defined by a mounting plate (e.g., 38, 40, 42) can define internal threads that are sized and configured to mate with a fastener exterior thread, as described in more detail herein.

In the illustrated embodiment, the plurality of fasteners 18 includes a first fastener 76, a second fastener 78, and a third fastener 80. As shown in FIGS. 1 and 2, examples of suitable fasteners to include in a plurality of fasteners 18 include screws 82. Each screw 82 has a fastener first end 84, a fastener second end 86, a fastener length 87, and a fastener main body 88 that defines a fastener head 90 and fastener external threads 92. The fastener length 87 extends from the fastener first end 84 to the fastener second end 86 and the fastener external threads 92 extend along a portion of the fastener length 87 and are sized and configured to attach a fastener to a mounting plate (e.g., have a fastener length that is less than the combined thickness of a mounting plate and a mounting jaw or the combined thickness of a mounting plate and a mounting clamp such that the fastener does not extend into cartilage during use). In the illustrated embodiment, the fastener external threads 92 are self-tapping threads and the fastener length 87 of each fastener of the plurality of fasteners 18 is about ⅜ inches and is less than the combined thickness of the mounting plate thickness 27 and the mounting jaw thickness 47 and the combined thickness of the mounting plate thickness 27 and the mounting clamp thickness 61.

In the illustrated embodiment, the first fastener 76 is sized and configured to be received by the mounting jaw aperture 56 and the mounting plate first aperture 38, the second fastener 78 is sized and configured to be received by the mounting clamp first aperture 70 and the mounting plate second aperture 40, and the third fastener 80 is sized and configured to be received by the mounting clamp second aperture 72 and the mounting plate third aperture 42. A portion of the fastener external threads 92 of the first fastener 76 has an outside diameter 77 that is greater than the inside diameter of the mounting plate first aperture 38 and the mounting jaw aperture 56 such that a friction fit between the first fastener 76, the mounting plate 12, and the mounting jaw 14 is created when the first fastener 76 is passed through the mounting jaw 14 and mounting plate 12. A portion of the fastener external threads 92 of the second fastener 78 has an outside diameter 79 that is greater than the inside diameter of the mounting plate second aperture 40 and the mounting clamp first aperture 70 such that a friction fit between the second fastener 78, the mounting plate 12, and the mounting clamp 16 is created when the second fastener 78 is passed through the mounting jaw 14 and mounting clamp 16. A portion of the fastener external threads 92 of the third fastener 80 has an outside diameter 81 that is greater than the inside diameter of the mounting plate third aperture 42 and the mounting clamp second aperture 72 such that a friction fit between the third fastener 80, the mounting plate 12, and the mounting clamp 16 is created when the third fastener 80 is passed through the mounting jaw 14 and mounting clamp 16.

In the illustrated embodiment, each of the fasteners 18 is formed of a first material and each of the mounting plate 12, mounting jaw 14, and mounting clamp 16 is formed of a second material that is different than the first material and relatively more flexible than the first material (e.g., the second material has a durometer hardness that is greater than the durometer hardness of the first material). While the first material that forms each of the fasteners 18 has been described as different than the second material that forms each of the mounting plate 12, mounting jaw 14, and mounting clamp 16, each of the fasteners, mounting plate, mounting jaw, mounting clamp of a medical device can be formed of any suitable material and selection of a suitable material can be based on various considerations, such as the material forming the portion of the body to which the medical device is intended to be attached (e.g., thyroid cartilage). For example, a first material that forms a fastener can be the same as, or different than, a second material that forms a mounting plate, mounting jaw, and/or mounting clamp.

While each fastener the plurality of fasteners has been described as forming a friction fit between a mounting plate, mounting jaw, and/or mounting clamp, alternative embodiments can include a mounting plate, mounting jaw, and/or mounting clamp that include threads sized and configured to mate with the threads of a fastener. For example, the mounting plate first aperture of a mounting plate and the mounting jaw aperture of a mounting jaw can define internal threads that are sized and configured to mate with the fastener external threads of a first fastener, the mounting plate second aperture of a mounting plate and the mounting clamp first aperture of a mounting clamp can define internal threads that are sized and configured to mate with the fastener external threads of a second fastener, and/or the mounting plate third aperture of a mounting plate and the mounting clamp second aperture of a mounting clamp can define internal threads that are sized and configured to mate with the fastener external threads of a third fastener. Determining whether a mounting plate, mounting jaw, and/or mounting clamp should include internal threads in an aperture can be based on various considerations, such as the material that forms the mounting plate, mounting jaw, mounting clamp, and/or fastener. In embodiments in which a mounting plate defines a fourth aperture and a fifth aperture, the mounting plate fourth aperture and/or mounting plate fifth aperture can define internal threads that are sized and configured to mate with the fastener external threads of a fourth fastener and/or fifth fastener.

While the medical device 10 is illustrated as including first, second, and third fasteners 76, 78, and 80, a medical device can include any suitable number of fasteners. Example numbers of fasteners considered suitable to include in a medical device include zero, one, at least one, two, a plurality, three, four, five, six, seven, and any other number considered suitable for a particular embodiment. The number of fasteners included in a medical device will depend on the number of apertures defined by a mounting plate, a mounting jaw, and/or mounting clamp and/or the structural configuration of the portion of the body to which the mounting plate is intended to be attached (e.g., thyroid cartilage). For example, in embodiments in which the mounting plate defines fourth and mounting plate fifth apertures, a plurality of fasteners can include a fourth fastener and a fifth fastener that are each sized and configured to be received within an aperture defined by the mounting plate to assist with attachment to the portion of the body to which the mounting plate is intended to be attached (e.g., thyroid cartilage). Alternatively, a medical device can omit the inclusion of a fastener, or a plurality of fasteners, and include a mounting plate, mounting jaw, and/or mounting clamp.

While the first, second, and third fasteners 76, 78, and 80 have been illustrated as having a particular structural arrangement, a fastener included in a medical device can have any suitable shape, size, and configuration. The shape, size, and configuration of a fastener included in a medical device will depend on various considerations, such as the thickness of a mounting plate, mounting jaw, and/or mounting clamp, the configuration of the apertures defined by a mounting plate, mounting jaw, and/or mounting clamp, and/or the structural configuration of the wall or feature to which a mounting plate included in a medical device is intended to be attached.

The medical device 10 is particularly well-suited for use with a tissue manipulator, such as in the example methods described herein. For example, a tissue manipulator can be disposed through the mounting plate passageway 36 and releasably attached to the mounting plate 12. The mounting jaw 14 can be releasably attached to the mounting plate 12 with the first fastener 76. The mounting clamp 16 can be releasably attached to the mounting plate 12 with the second fastener 78 and third fastener 80. When assembled, the tissue manipulator is partially disposed between the mounting plate 12 and mounting jaw 14 and between the mounting plate 12 and the mounting clamp 16. The mounting plate 12 can be attached to tissue, such as patient tissue engaged by the tissue manipulator or tissue adjacent or near patient tissue engaged by the tissue manipulator, with fasteners of the plurality of fasteners (e.g., fourth fastener, fifth fastener).

Each of the mounting plate 12, mounting jaw 14, mounting clamp 16, and each fastener of the plurality of fasteners 18 can be formed of any suitable material and selection of a suitable material to form a mounting plate, a mounting jaw, a mounting clamp, and each fastener of a plurality of fasteners according to a particular embodiment can be based on various considerations, including the portion of the body (e.g., thyroid cartilage) to which a medical device is intended to be attached. Example materials considered suitable to form a mounting plate, a mounting jaw, a mounting clamp, and a plurality of fasteners include rigid materials, semi-rigid materials, implant grade materials, implant grade rigid materials, implant grade semi-rigid materials, biocompatible materials, materials that can be made biocompatible, metals such as stainless steel, titanium, metal alloys, nickel-titanium alloy (e.g., Nitinol), thermoplastics, polymers, polyetheretherketone (PEEK), Pebax (Pebax is a registered trademark of Ato Chimie Corporation of Allee des Vosges, Courbevoie, France), nylon, polyethylene, high-density polyethylene (HDPE), high-performance polyethylene (HPPE), polyurethane, silicone, acrylonitrile butadiene styrene (ABS), polyoxymethylene (e.g., acetal), combinations of those described herein, and any other material considered suitable for a particular embodiment. In the illustrated embodiment, each of the mounting plate 12, mounting jaw 14, and mounting clamp 16 is formed of the same malleable material that can be formed by a physician's hands prior to, or during, implantation or by the anatomy of the patient to conform to the patient's anatomy and each fastener of the plurality of fasteners 18 is formed of stainless steel.

While each of the mounting plate 12, mounting jaw 14, and mounting clamp 16 has been described as being formed of the same malleable material and each fastener of the plurality of fasteners 18 has been described as being formed of stainless steel, a mounting plate, a mounting jaw, a mounting clamp, and each fastener of a plurality of fasteners included in a medical device can be formed of any suitable material. Selection of a suitable material to form a mounting plate, a mounting jaw, a mounting clamp, and each fastener of a plurality of fasteners included in a medical device according to a particular embodiment can be based on various considerations such as the portion of the body to which the medical device is intended to be attached (e.g., thyroid cartilage). For example, each of the mounting plate, mounting jaw, mounting clamp, and each fastener of a plurality of fasteners can be formed of the same material. Alternatively, the mounting plate can be formed of a first material, a mounting jaw can be formed of a second material, a mounting clamp can be formed of a third material, and each fastener included in a plurality of fasteners, or a set of fasteners included in a plurality of fasteners, can be formed of a fourth material. The first material can be the same as, or different than, the second material, third material, and/or fourth material. The second material can be the same as, or different than, the first material, third material, and/or fourth material. The third material can be the same as, or different than, the first material, second material, and/or fourth material. The fourth material can be the same as, or different than, the first material, second material, and/or third material.

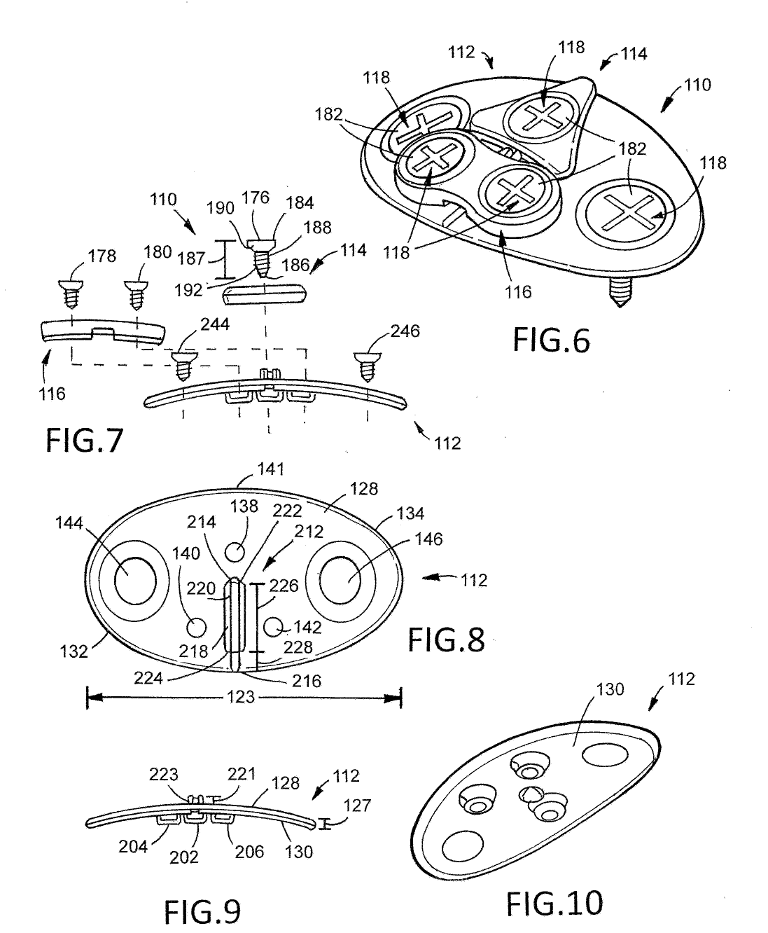

FIGS. 6, 7, 8, 9, 10, 11, 12, 13, 14, and 15 illustrate another example medical device 110. The medical device 110 is similar to the medical device 10 illustrated in FIGS. 1, 2, 3, 4, and 5 and described above, except as detailed below. The medical device 110 comprises a mounting plate 112, a mounting jaw 114, a mounting clamp 116, and a plurality of fasteners 118.

In the illustrated embodiment, each of the mounting plate front surface 128 and the mounting plate back surface 130 has a pre-formed curve that extends across the entire mounting plate length 123. This structural configuration provides a mounting plate front surface 128 and a mounting plate back surface 130 that conforms to the anatomy of the patient and that is sized and configured to mate with the portion of the body to which the mounting plate 112 is intended to be attached (e.g., thyroid cartilage). In the illustrated embodiment, the mounting plate 112 is an elongated cylinder and the mounting plate main body 134 defines a mounting plate fourth aperture 144, a mounting plate fifth aperture 146, a mounting plate first projection 202, a mounting plate second projection 204, a mounting plate third projection 206, and a mounting plate track 212. The mounting plate 112 has a substantially constant mounting plate thickness 127 not including the thickness at the mounting plate first projection 202, the mounting plate second projection 204, the mounting plate third projection 206, and the mounting plate track 212. The mounting plate first projection 202 extends around the entire circumference of the mounting plate first aperture 138 from the mounting plate back surface 130 and away from the mounting plate front surface 128. The mounting plate second projection 204 extends around the entire circumference of the mounting plate second aperture 140 from the mounting plate back surface 130 and away from the mounting plate front surface 128. The mounting plate third projection 206 extends around the entire circumference of the mounting plate third aperture 142 from the mounting plate back surface 130 and away from the mounting plate front surface 128. Each of the projections 202, 204, 206 has a rounded, blunted, atraumatic end. While each projection 202, 204, 206 has been illustrated as extending around the entire circumference of an aperture, alternative embodiments can include a mounting plate projection that extends around a portion of the circumference of an aperture and/or a mounting plate that omits the inclusion of a first, second, and third projection extending from the mounting plate back surface and away from the mounting plate front surface. When included, mounting plate projections allow for a mounting plate to have a thin profile and allow for increased securement between the mounting plate and the structure to which it is being attached.

Each of the mounting plate fourth aperture 144 and mounting plate fifth aperture 146 extends from the mounting plate front surface 128 to the mounting plate back surface 130 and is sized and configured to receive a fastener of the plurality of fasteners 118 (e.g., has a diameter that is greater than the major diameter of a fastener intended to be passed through the aperture). In the illustrated embodiment, each of the mounting plate fourth aperture 144 and mounting plate fifth aperture 146 is disposed on a first hypothetical plane that contains the center of the mounting plate 112 and is disposed orthogonal to a second hypothetical plane that contains the mounting plate passageway 136 and the mounting plate track recess 220, as described in more detail herein. In addition, each of the mounting plate fourth aperture 144 and mounting plate fifth aperture 146 is directed inward, toward the center of the mounting plate 112, from the mounting plate front surface 128 toward the mounting plate back surface 130 such that the lengthwise axis of each aperture is disposed at an angle to a first hypothetical plane that contains the mounting plate passageway 136 and the mounting plate track 212. While the mounting plate fourth aperture 144 and the mounting plate fifth aperture 146 have been illustrated as positioned at a particular location on the mounting plate 112 and directed toward the center of the mounting plate 112, a mounting plate aperture can be positioned at any suitable location on a mounting plate and selection of a suitable location can be based on various considerations, such as the structural configuration of a mounting jaw and/or mounting clamp included in a medical device. Example locations considered suitable to position a mounting plate aperture on a mounting plate include positioning a mounting plate fourth aperture on a first hypothetical plane that contains the center of the mounting plate and is disposed orthogonal to a second hypothetical plane that contains a mounting plate passageway and a mounting plate track recess and a mounting plate fifth aperture on a third hypothetical plane that does not contain the center of the mounting plate and is disposed parallel to the first hypothetical plane such that the fourth and mounting plate fifth apertures are offset from one another relative to the center of the mounting plate. Alternative to a mounting plate aperture being directed toward the center of a mounting plate, a mounting plate aperture can have a lengthwise axis that is disposed orthogonal to a mounting plate front surface and/or mounting plate back surface.

In the illustrated embodiment, the mounting plate track 212 comprises a mounting plate track first end 214, a mounting plate track second end 216, a mounting plate track projection 218 and a mounting plate track recess 220 that has a portion cooperatively defined by the mounting plate track projection 218 and mounting plate main body 134. In the illustrated embodiment, the mounting plate passageway 136, the mounting plate first aperture 138, and the mounting plate track 212 are disposed on a hypothetical plane 141 that extends through the mounting plate main body 134 and between the mounting plate second aperture 140 and mounting plate third aperture 142. When assembled, a tissue manipulator is partially disposed within the mounting plate track 212 (e.g., mounting plate track recess 220).

The mounting plate projection 218 extends from the mounting plate front surface 128 and away from the mounting plate back surface 130 and has a mounting plate track first end 222, a mounting plate track second end 224, a first mounting plate track projection height 221, and a second mounting plate track projection height 223. The mounting plate track first end 222 is disposed between the mounting plate passageway 136 and the mounting plate side 132 and between the mounting plate second aperture 140 and the mounting plate third aperture 142. The mounting plate track second end 224 is disposed between the mounting plate track first end 222 and the mounting plate side 132. Alternative embodiments, however, can include a mounting plate track that extends to a mounting plate side such that the mounting plate track second end is adjacent to, or coplanar with, the mounting plate side. The first mounting plate track projection height 221 is disposed at the mounting plate track first end 222 and the second mounting plate track projection height 223 is disposed at the mounting plate track second end 224. The first mounting plate track projection height 221 is greater than the second mounting plate track projection height 223 and the mounting plate track projection height tapers from the mounting plate track first end 222 to the mounting plate track second end 224.

The mounting plate track recess 220 is sized and configured to receive a portion of a tissue manipulator, as described in more detail herein, and has a mounting plate track recess first portion 226 and a mounting plate track recess second portion 228. The mounting plate track recess first portion 226 extends into the mounting plate track projection 218 from the mounting plate track first end 222 to the mounting plate track second end 224. The mounting plate track recess second portion 228 extends into the mounting plate main body 134 from the mounting plate track projection second end 224 to the mounting plate side 132. Thus, the mounting plate track 212 extends along a portion of the mounting plate width 125 and a portion of the mounting plate length 123 and from a mounting plate projection first end 222 disposed between the mounting plate passageway 136 and the mounting plate side 132 to the mounting plate side 132.

While the mounting plate track 212 has been described as having a particular structural configuration, a mounting plate track included on a mounting plate can have any suitable structural configuration and selection of a suitable structural configuration can be based on various considerations, such as the structural configuration of a tissue manipulator intended to be used with a mounting plate. For example, an alternative mounting plate track can include first and second projections that extends from the mounting plate front surface and away from the mounting plate back surface and taper from the mounting plate track first end to the mounting plate track second end. The mounting plate track second end can be disposed at, or near, the mounting plate side and the mounting plate track recess can be defined by the raised projections or cooperatively by the raised projections and the mounting plate main body.

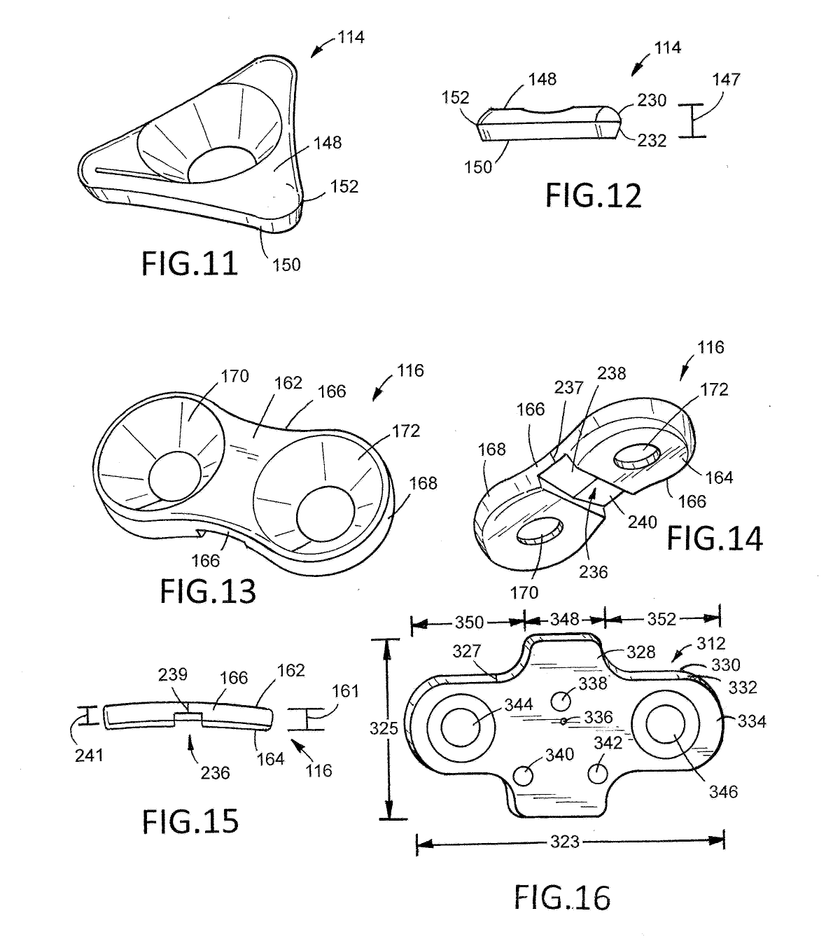

As shown in FIGS. 11 and 12, the mounting jaw front surface 148 is substantially flat (e.g., a majority, or entity, of the surface is disposed on a plane) and the mounting jaw side 152 defines a first mounting jaw surface 230 and a second mounting jaw surface 232. The first mounting jaw surface 230 extends from the mounting jaw front surface 148 to the second mounting jaw surface 232 and the second mounting jaw surface 232 extends from the first mounting jaw surface 230 to the mounting jaw back surface 150. In the illustrated embodiment, the first mounting jaw surface 230 is curved and the second mounting jaw surface is flat and tapers from the first mounting jaw surface to the mounting jaw back surface 150.

In the illustrated embodiment, the mounting clamp 116 is partially disposed over the mounting plate track 212, and, as shown in FIGS. 13, 14, and 15, each of the mounting clamp front surface 162 and the mounting clamp back surface 164 is curved, and the mounting clamp main body 168 defines a mounting clamp recess 236. The curvature of the mounting clamp front surface 162 and mounting clamp back surface 164 provides a mechanism for reducing trauma to tissue during, and subsequent, to implantation of the medical device in a patient and provides a mechanism for receiving a portion of a tissue manipulator within the mounting clamp recess 236.

The mounting clamp recess 236 is disposed between the mounting clamp first aperture 170 and the mounting clamp second aperture 172 and has a mounting clamp recess first portion 238 and a mounting clamp recess second portion 240. The mounting clamp recess first portion 238 extends from the mounting clamp side 166 to the mounting clamp recess second portion 240 and the mounting clamp recess second portion 240 extends from the mounting clamp side 166 to the mounting clamp recess first portion 238. As shown in FIGS. 14 and 15, the mounting clamp 116 has a first mounting clamp thickness 237 at the mounting clamp side 166 and within the mounting clamp recess first portion 238, a second mounting clamp thickness 239 at the mounting clamp side 166 and within the mounting clamp recess second portion 240, and a third mounting clamp thickness 241 at the location where the mounting clamp recess first portion 238 meets the mounting clamp recess second portion 240. The third mounting clamp thickness 241 is greater than the first mounting clamp thickness 237 and the second mounting clamp thickness 239. The variation in the mounting clamp thickness provides a mechanism for releasably attaching a tissue manipulator between the mounting plate 112 and the mounting clamp 116.

In the illustrated embodiment, the plurality of fasteners 118 includes a first fastener 176, a second fastener 178, a third fastener 180, a fourth fastener 244, and a fifth fastener 246. As shown in FIGS. 6 and 7, examples of suitable fasteners to include in a plurality of fasteners 118 include screws 182. Each screw 182 has a fastener first end 184, a fastener second end 186, a fastener length 187, and a fastener main body 188 that defines a fastener head 190 and fastener external threads 192. In the illustrated embodiment, the fastener length 187 of each fastener 244, 246 is greater than the combined thickness of the mounting plate thickness 127 and the mounting jaw thickness 147 and the combined thickness of the mounting plate thickness 127 and the mounting clamp thickness 161. In the illustrated embodiment, each fastener 176, 178, 180 has a first structural configuration (e.g., similar to a machine screw) and each fastener 244, 246 has a second structural configuration (e.g., is self-tapping). However, alternative embodiments can include a first set of fasteners that have the same structural configuration as a second set of fasteners.

A portion of the fastener external threads 192 of the fourth fastener 244 has an outside diameter that is greater than the inside diameter of the mounting plate fourth aperture 144 such that a friction fit between the fourth fastener 244 and the mounting plate 112 is created when the fourth fastener 244 is passed through the mounting plate 12. A portion of the fastener external threads 192 of the fifth fastener 246 has an outside diameter that is greater than the inside diameter of the mounting plate fifth aperture 146 such that a friction fit between the fifth fastener 246 and the mounting plate 12 is created when the fifth fastener 246 is passed through the mounting plate 112. When being implanted, the fastener external threads 192 of each of the fourth and fifth fasteners 244, 246 can have an outside diameter that is greater than the inside diameter of a pre-drilled pilot hole in the portion of the body to which the mounting plate 112 is intended to be attached (e.g., thyroid cartilage) when a pilot hole has been created.

While each of the fourth and fifth fasteners 244, 246 has been described as forming a friction fit between the fasteners and the mounting plate 112, alternative embodiments can include a mounting plate that defines a fourth aperture and a fifth aperture that each include internal threads that are sized and configured to mate with the external threads of a fastener. For example, the mounting plate fourth aperture and/or mounting plate fifth aperture of a mounting plate can define internal threads that are sized and configured to mate with the fastener external threads of a fourth fastener and/or fifth fastener. Determining whether a mounting plate should include internal threads in an aperture can be based on various considerations, such as the material that forms the mounting plate and/or fastener.

While medical device 110 is illustrated as including first, second, third, fourth, and fifth fasteners 176, 178, 180, 244, 246, a medical device can include any suitable number of fasteners. Example numbers of fasteners considered suitable to include in a medical device include one, at least one, two, a plurality, three, four, five, six, seven, and any other number considered suitable for a particular embodiment. The number of fasteners included in a medical device will depend on the number of apertures defined by a mounting plate, a mounting jaw, and/or mounting clamp and/or the structural configuration of the portion of the body to which the medical device is intended to be attached.

The medical device 110 is particularly well-suited for use with a tissue manipulator, such as in the example methods described herein. For example, a tissue manipulator can be disposed through the mounting plate passageway 136 and releasably attached to the mounting plate 112. The mounting jaw 114 can be releasably attached to the mounting plate 112 with the first fastener 176. The mounting clamp 116 can be releasably attached to the mounting plate 112 with the second fastener 178 and third fastener 180. When assembled, the tissue manipulator is partially disposed between the mounting plate 112 and mounting jaw 114 and between the mounting plate 112 and the mounting clamp 116. In addition, when assembled, a portion of the tissue manipulator is disposed in the mounting plate track 212 (e.g., mounting plate track recess 220) and within the mounting clamp recess 236. The mounting plate 112 can be attached to tissue, such as patient tissue engaged by the tissue manipulator or tissue adjacent or near patient tissue engaged by the tissue manipulator with the fourth fastener 244 and the fifth fastener 246.

Optionally, a mounting jaw and/or mounting clamp can be preloaded on the medical device to reduce the complexity of, and time required to complete, a procedure. With respect to the mounting jaw, this can be accomplished, for example, by partially passing a fastener through the mounting jaw aperture and into the mounting plate first aperture. With respect to the mounting clamp, this can be accomplished, for example, by partially passing a fastener through the mounting clamp first aperture and into the mounting plate second aperture and/or the mounting clamp second aperture and in to the mounting plate third aperture.

FIG. 16 illustrates an alternative mounting plate 312 suitable for inclusion in a medical device according to an embodiment. The mounting plate 312 is similar to the mounting plate 12 illustrated in FIGS. 1, 2, and 3 and described above, except as detailed below. In the illustrated embodiment, the mounting plate 312 has a mounting plate length 323, a mounting plate width 325, a mounting plate thickness 327, a mounting plate front surface 328, a mounting plate back surface 330, a mounting surface side 332, and a mounting plate main body 334 that defines a mounting plate passageway 336, a mounting plate first aperture 338, a mounting plate second aperture 340, a mounting plate third aperture 342, a mounting plate fourth aperture 344, a mounting plate fifth aperture 346, a mounting plate first portion 348, a first mounting plate extended member 350, and a second mounting plate extended member 352.

The mounting plate first portion 348 is disposed between the first mounting plate extended member 350 and the second mounting plate extended member 352. Each of the first mounting plate extended member 350 and second mounting plate extended member 352 extends from the mounting plate first portion 348 and away from the mounting plate passageway 336. The mounting plate fourth aperture 344 is disposed on the first mounting plate extended member 350 and the mounting plate fifth aperture 346 is disposed on the second mounting plate extended member 352. This structural arrangement positions the mounting plate fourth aperture 344 between the mounting plate side 332 and a mounting jaw and mounting plate fifth aperture 346 between the mounting plate side 332 and a mounting jaw when the medical device is assembled.

While a particular structural configuration has been illustrated for the mounting plate 312, as described herein, a mounting plate can have any suitable structural configuration. For example, alternative embodiments of a mounting plate, such as the mounting plate 312 described in FIG. 16, can include a curved mounting plate front surface and/or mounting plate back surface, a mounting plate track, such as those described herein (e.g., 212, 412, 712).

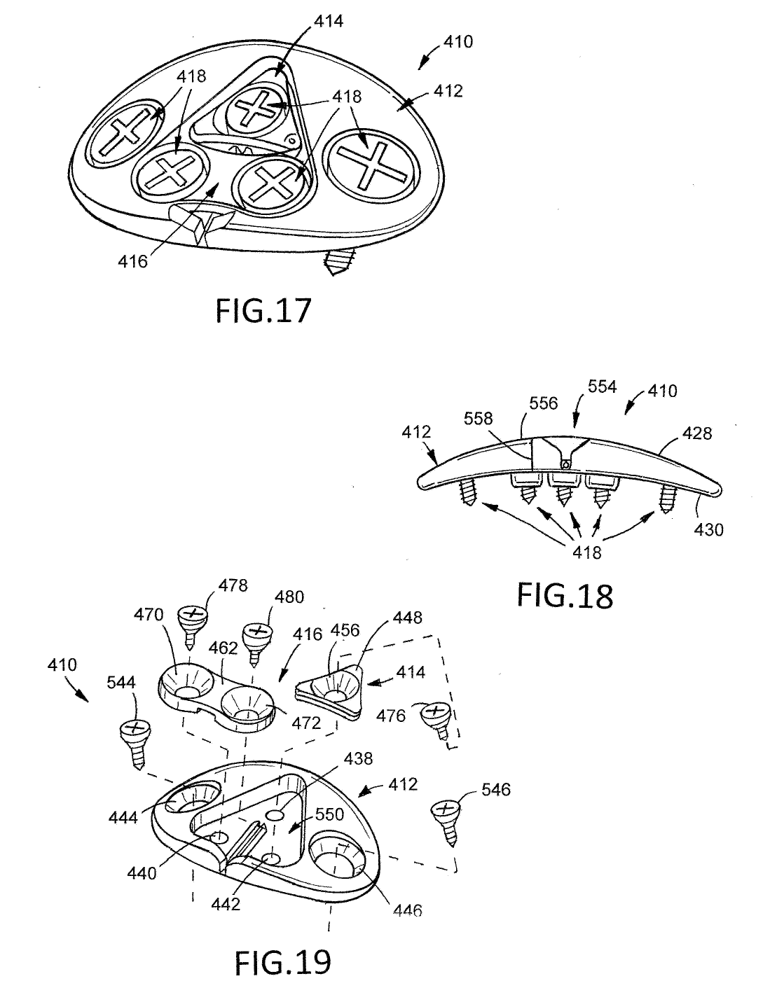

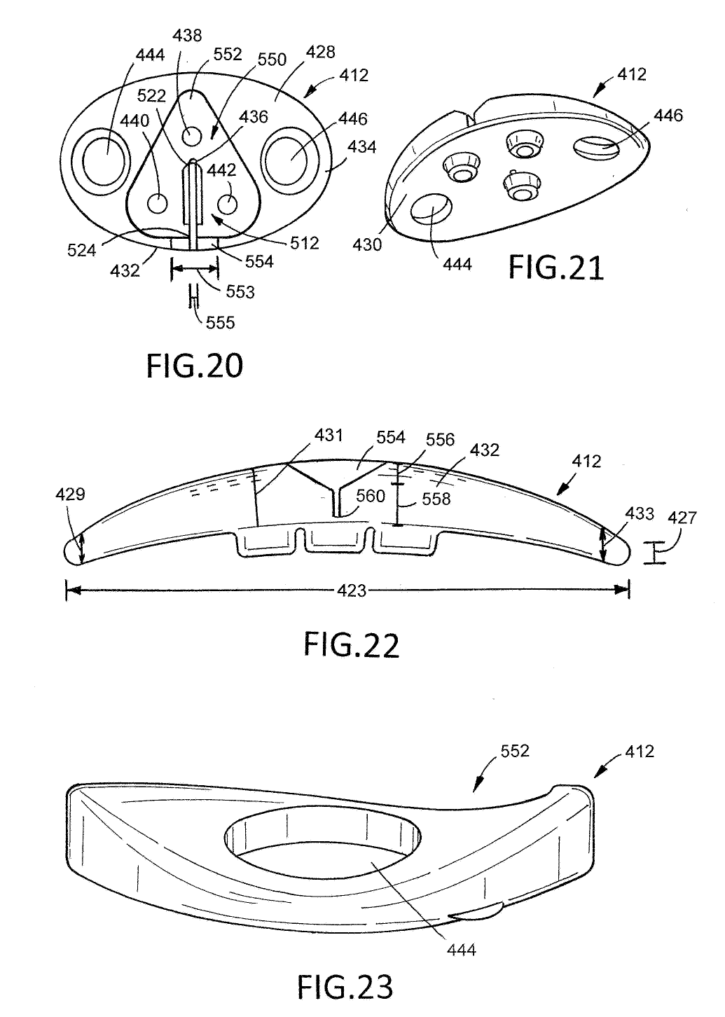

FIGS. 17, 18, 19, 20, 21, 22, and 23 illustrate another example medical device 410. The medical device 410 is similar to the medical device 110 illustrated in FIGS. 6, 7, 8, 9, 10, 11, 12, 13, 14, and 15 and described above, except as detailed below. The medical device 410 comprises a mounting plate 412, a mounting jaw 414, a mounting clamp 416, and a plurality of fasteners 418.

As shown in FIG. 22, the mounting plate thickness 427 varies along both the mounting plate length 423 and the mounting plate width 425. The mounting plate 412 has a first mounting plate thickness 429, a second mounting plate thickness 431, and a third mounting plate thickness 433. The first mounting plate thickness 429 is disposed between the mounting plate side 432 and the mounting plate fourth aperture 444, the second mounting plate thickness 431 is disposed between the first mounting plate thickness 429 and the third mounting plate thickness 433 and between the mounting plate fourth aperture 444 and the mounting plate fifth aperture 446, and the third mounting plate thickness 433 is disposed between the mounting plate side 432 and the mounting plate fifth aperture 446. The second mounting plate thickness 431 is greater than both the first mounting plate thickness 429 and the third mounting plate thickness 433.

In the illustrated embodiment, the mounting plate main body 434 defines a mounting plate recess 550 and a mounting plate channel 554. The mounting plate recess 550 extends from the mounting plate front surface 428, into the mounting plate main body 434, and toward the mounting plate back surface 430 to a mounting plate recess base 552 and has a triangular prismatic shape. The mounting plate recess 550 is sized and configured to receive each of the mounting jaw 414 and mounting clamp 416 such that the mounting jaw front surface 448 and the mounting clamp front surface 462 are disposed within the mounting plate recess 550 when releasably attached to the mounting plate 412, as shown in FIG. 18. In the illustrated embodiment, each of the mounting plate passageway 436, the mounting plate first aperture 438, the mounting plate second aperture 440, and the mounting plate third aperture 442 is disposed within the mounting plate recess 550 and extends from the mounting plate recess base 552 to the mounting plate back surface 430. This structural configuration provides a mechanism for reducing the overall thickness of the medical device 410 relative to those medical devices (e.g., 10, 110) that do not position a mounting jaw and/or mounting clamp within a mounting plate recess and for providing a relatively continuous medical device front surface when the mounting plate 412, the mounting jaw 414, the mounting clamp 416, and the plurality of fasteners 418 have been assembled. In the illustrated embodiment, the mounting jaw 414 and mounting clamp 416 are entirely disposed within the mounting plate recess 550 such that they do not protrude past a hypothetical surface that extends along the mounting plate front surface 448 and across the mounting plate recess 550.

The mounting plate channel 554 extends from the mounting plate side 432 to the mounting plate recess 550, provides access to the mounting plate recess 550, and is sized and configured to receive a portion of a tissue manipulator, such as those described herein. The mounting plate channel 554 has a first mounting plate channel portion 556, a second mounting plate channel portion 558, and a mounting plate channel base 560. The first mounting plate channel portion 556 has a first mounting plate channel length 553 and the second mounting plate channel portion 558 has a second mounting plate channel length 555 that is less than the first mounting plate channel length 553. The first mounting plate channel length 553 is measured along a first hypothetical surface that extends along the mounting plate front surface 428 and over the mounting plate channel 554 and the second mounting plate channel length 555 is measured along a second hypothetical surface that extends between the first hypothetical surface and the mounting plate back surface 430. As shown in FIG. 18, the first mounting plate channel portion 556 tapers from the mounting plate top surface 428 toward the mounting plate back surface 430 to the second mounting plate channel portion 558, which has a constant length from the first mounting plate channel portion 556 to the channel base 560.

In the illustrated embodiment, the mounting plate track 512 is disposed within the mounting plate recess 550 such that the mounting plate track first end 522 is disposed between the mounting plate passageway 436 and the mounting plate side 432 and the mounting plate track second end 524 is disposed at the junction between the mounting plate recess 550 and the mounting plate channel 554.

The medical device 410 is particularly well-suited for use with a tissue manipulator, such as in the example methods described herein. For example, a tissue manipulator can be disposed through the mounting plate passageway 436 and releasably attached to the mounting plate 412. The mounting jaw 414 can be releasably attached to the mounting plate 412 with the first fastener 476. The mounting clamp 416 can be releasably attached to the mounting plate 412 with the second fastener 478 and third fastener 480. When assembled, the tissue manipulator is partially disposed between the mounting plate 412 and mounting jaw 414 and between the mounting plate 412 and the mounting clamp 416. In addition, when assembled, a portion of the tissue manipulator is disposed in the mounting plate track 512 (e.g., mounting plate track recess), the mounting plate channel 554, and within the mounting clamp recess. The mounting plate 412 can be attached to tissue, such as patient tissue engaged by the tissue manipulator or tissue adjacent or near patient tissue engaged by the tissue manipulator, with the fourth fastener 544 and fifth fastener 546.

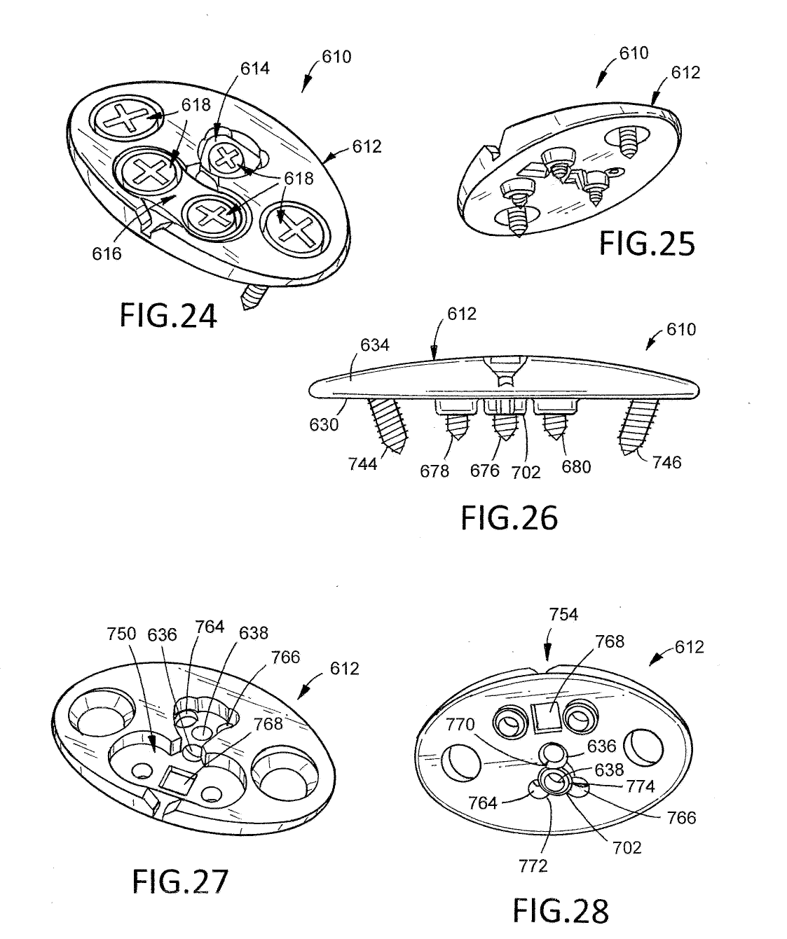

FIGS. 24, 25, 26, 27, 28, 29, 30, 31, 32, and 33 illustrate another example medical device 610. The medical device 610 is similar to the medical device 410 illustrated in FIGS. 17, 18, 19, 20, 21, 22, and 23 and described above, except as detailed below. The medical device 610 comprises a mounting plate 612, a mounting jaw 614, a mounting clamp 616, and a plurality of fasteners 618.

In the illustrated embodiment, the mounting plate back surface 630 is flat and the mounting plate main body 634 defines a first mounting plate passageway 636, a mounting plate second passageway 764, a mounting plate third passageway 766, a mounting plate fourth passageway 768, a mounting plate first projection first recess 770, a mounting plate second projection recess 772, and a mounting plate third projection recess 774. In the illustrated embodiment, the first mounting plate passageway 636 has an inside diameter that is greater than the mounting plate passageways described in other example medical devices such that it is sized and configured to receive a mounting jaw projection (e.g., 776, 778, 780) and a portion of a tissue manipulator, as described in more detail herein. In addition, the mounting plate passageway 636 is sized and configured to receive a portion of a delivery cannula such that a physician can alter the position of, or remove, a tissue manipulator after the medical device has been implanted. In the illustrated embodiment, the mounting plate passageway 636 has an inside diameter that is greater than the maximum coil turn outside diameter of a tissue manipulator, as described in more detail herein. While mounting plate back surface 630 has been illustrated as flat, it can alternatively define a predefined curve along a portion, or the entirety, of the mounting plate length, as described in more detail herein.

The mounting plate recess 750 has a configuration that corresponds to the configuration of the mounting jaw 614 and the mounting clamp 616. Each of the first mounting plate passageway 636, mounting plate second passageway 764, mounting plate third passageway 766 is disposed adjacent to the mounting plate first aperture 638 and is sized and configured to receive a projection of the plurality of mounting jaw projections 776 defined by the mounting jaw 614. The mounting plate fourth passageway 768 is disposed between the first mounting plate passageway 636 and the mounting plate channel 754 and is sized and configured to receive a portion of the mounting clamp projection 790.

Each of the mounting plate first projection first recess 770, the mounting plate first projection second recess 772, and the mounting plate first projection third recess 774 is disposed on the mounting plate first projection 702. The mounting plate first projection recess 770 is disposed adjacent the first mounting plate passageway 636, the mounting plate second projection recess 772 is disposed adjacent the mounting plate second passageway 764, and the mounting plate third projection recess is disposed adjacent the mounting plate third passageway 766. Each of the mounting plate first projection recess 770, the mounting plate second projection recess 772, and the mounting plate third projection recess 774 is sized and configured to receive a portion of a projection of the plurality of mounting jaw projections 776, as described in more detail herein.

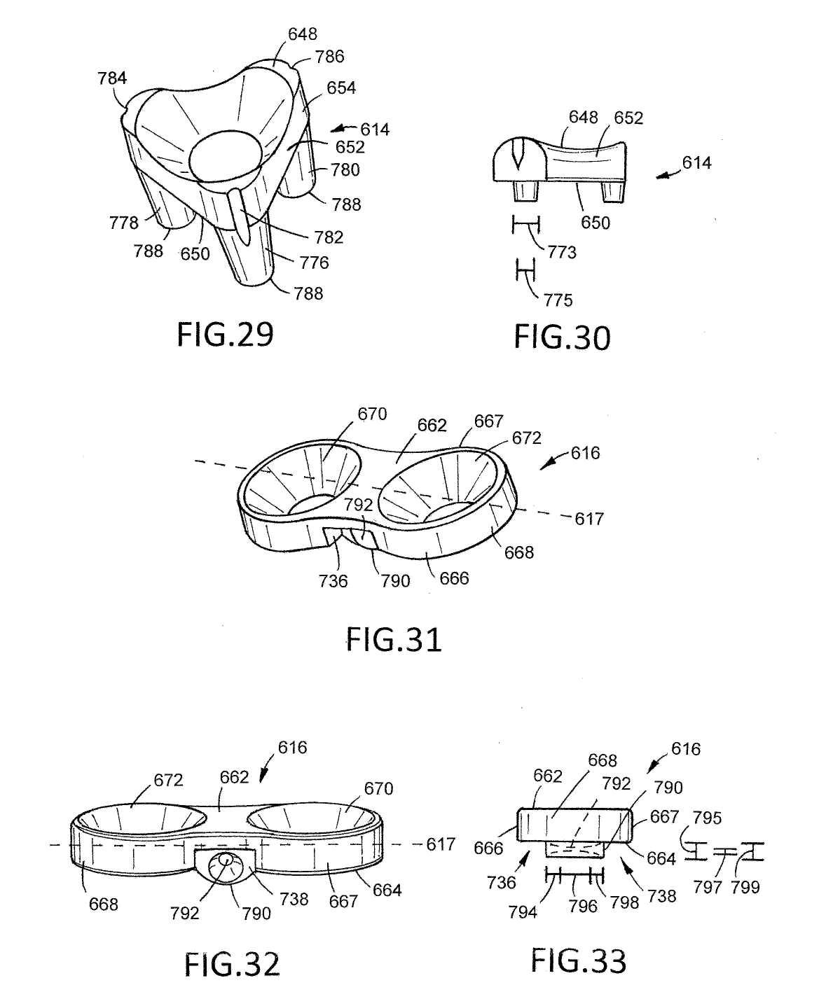

As shown in FIGS. 29 and 30, the mounting jaw main body 654 defines a first mounting jaw projection 776, a second mounting jaw projection 778, a third mounting jaw projection 780, a first mounting jaw recess 782, a second mounting jaw recess 784, and a third mounting jaw recess 786. Each of the mounting jaw projections 776, 778, 780 extends from the mounting jaw back surface 650 and away from the mounting jaw front surface 648 to a mounting jaw projection end 788. Each of the mounting jaw projections 776, 778, 780 has a first mounting jaw projection outside diameter 773 at the mounting jaw back surface 650 and a second mounting jaw projection outside diameter 775 at the mounting jaw projection end 778. The second mounting jaw projection outside diameter 775 is less than the first mounting jaw projection outside diameter 773 such that the outside diameter of each of the mounting jaw projections 776, 778, 780 tapers from the mounting jaw back surface 630 to the mounting jaw projection end 778. A portion of each of the mounting jaw projections 776, 778, 780 is sized and configured to be received by one of the first mounting plate passageway 636, mounting plate second passageway 764, or mounting plate third passageway 766, as described in more detail herein.

Each of the first mounting jaw recess 782, the second mounting jaw recess 784, and third mounting jaw recess 786 extends along the mounting jaw side 652 from the mounting jaw front surface 648 toward the mounting jaw back surface 650. Each of the first mounting jaw recess 782, the second mounting jaw recess 784, and third mounting jaw recess 786 is sized and configured to receive a portion of a tissue manipulator, as described in more detail herein, and provides a mechanism for maintaining the position of, and clamping, a tissue manipulator between the mounting jaw 614 and the mounting plate 612. This structural arrangement also provides a mechanism for filling any voids (e.g., entirely, or partially) in the mounting plate passageway 636 such that movement of a tissue manipulator that is disposed in the mounting plate passageway 636 can be avoided. The first mounting jaw recess 782 partially extends into the first mounting jaw projection 776, the second mounting jaw recess 784 partially extends into the second mounting jaw projection 778, and the third mounting jaw recess 786 partially extends into the third mounting jaw projection 780.

While the mounting jaw main body 654 has been illustrated as defining a first mounting jaw projection 776, a second mounting jaw projection 778, a third mounting jaw projection 780, a first mounting jaw recess 782, a second mounting jaw recess 784, and a third mounting jaw recess 786, a mounting jaw included in a medical device can include any suitable number of mounting jaw projections and/or mounting jaw recesses. Selection of a suitable number of mounting jaw projections and/or mounting jaw recesses can be based on various considerations, including the structural configuration of a mounting plate. Examples of numbers of mounting jaw projections and/or mounting jaw recesses considered suitable to include on a mounting jaw include zero, one, at least one, two, a plurality, three, four, five, and any other number considered suitable for a particular embodiment.

In the illustrated embodiment, the mounting clamp 618 has a lengthwise axis 617 and the mounting clamp main body 668 defines a first mounting clamp recess 736, a second mounting clamp recess 738, a mounting clamp projection 790, and a mounting clamp passageway 792. Each of the first mounting clamp recess 736 and second mounting clamp recess 738 is disposed between the mounting clamp first aperture 670 and the mounting clamp second aperture 672. Each of the first mounting clamp recess 736 and second mounting clamp recess 738 extends from the mounting clamp back surface 664, into the mounting clamp main body 668, and toward the mounting clamp front surface 662. The first mounting clamp recess 736 extends from a first mounting clamp side 666 and toward the mounting clamp lengthwise axis 617 and the second mounting clamp recess 738 extends from a second mounting clamp side 667 and toward the mounting clamp lengthwise axis 617. The second mounting clamp side 667 is opposably facing the first mounting clamp side 666. Alternative embodiments can omit the inclusion of a first mounting clamp recess and/or second mounting clamp recess.

The mounting clamp projection 790 is disposed between the mounting clamp first aperture 670 and the mounting clamp second aperture 672 and between the first mounting clamp recess 736 and the second mounting clamp recess 738. The mounting clamp projection 790 extends from the mounting clamp back surface 664 and away from the mounting clamp front surface 662 and has a rounded end. As shown in FIG. 33, the mounting clamp passageway 792 extends through the mounting clamp projection 790 and has mounting clamp passageway first portion 794, a mounting clamp passageway second portion 796, a mounting clamp passageway third portion 798 and is sized and configured to receive a portion of a tissue manipulator. The mounting clamp passageway first portion 794 has a first inside diameter 795, the mounting clamp passageway second portion 796 is disposed between the mounting clamp passageway first portion 794 and the mounting clamp passageway third portion 798 and has a second inside diameter 797, and the mounting clamp passageway third portion 798 has a third inside diameter 799. The first inside diameter 795 is greater than the second inside diameter 797 and the second inside diameter 797 is less than the third inside diameter 799. The mounting clamp passageway 792 has a diameter that tapers along the mounting clamp passageway first portion 794 toward the mounting clamp passageway second portion 796 and tapers from the mounting clamp passageway third portion 798 toward the mounting clamp passageway second portion 796. This configuration provides an additional pinching point for releasably attaching a tissue manipulator to the medical device 610.

The inclusion of a mounting clamp passageway 792 is considered advantageous at least because it provides a mechanism for reducing the complexity of an implantation procedure. For example, the end of a tissue manipulator can be passed through the mounting clamp passageway 792 and the combined mounting clamp 616 and tissue manipulator can be positioned such that the mounting clamp 616 can be releasably attached to the mounting plate 612. This results in a physician having to manipulate two items during this portion of the implantation procedure, the combined mounting plate 616 and tissue manipulator and the fasteners.

The medical device 610 is particularly well-suited for use with a tissue manipulator, such as in the example methods described herein. For example, a tissue manipulator can be disposed through the mounting plate passageway 636 and releasably attached to the mounting plate 612. The mounting jaw 614 can be releasably attached to the mounting plate 612 with the first fastener 676. The mounting clamp 616 can be releasably attached to the mounting plate 612 with the second fastener 678 and third fastener 680. When assembled, the tissue manipulator is partially disposed between the mounting plate 612 and mounting jaw 614 and between the mounting plate 612 and the mounting clamp 616. In addition, when assembled, a portion of the tissue manipulator is disposed in the mounting plate recess 750 and through the mounting clamp passageway 792. The mounting plate 612 can be attached to tissue, such as patient tissue engaged by the tissue manipulator or tissue adjacent or near patient tissue engaged by the tissue manipulator with the fourth fastener 744 and fifth fastener 746.

Any of the medical devices described herein, such as medical device 10, medical device 110, medical device 410, and/or medical device 610, can be used in combination with any of the tissue manipulators described herein, such as tissue manipulator 808, tissue manipulator 808′, tissue manipulator 908, and/or tissue manipulator 1008, and in any suitable method of treatment, such as the methods described herein, and can be attached to any suitable part, tissue, and/or portion of the body of a patient (e.g., thyroid cartilage).

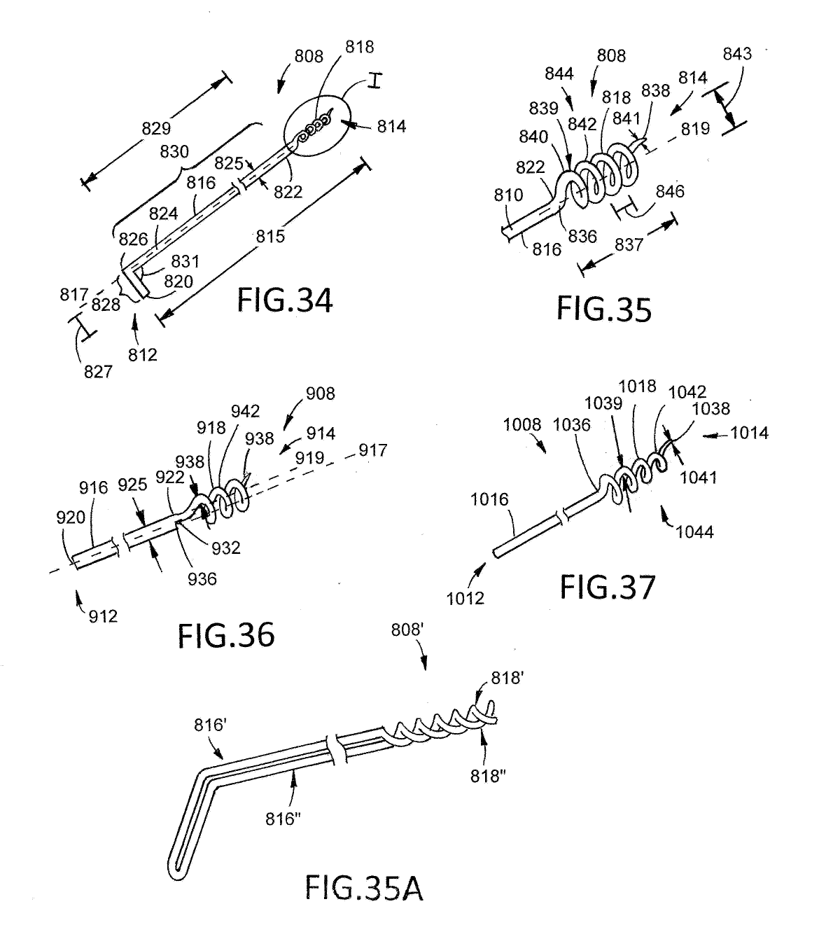

FIGS. 34 and 35 illustrate a first example tissue manipulator 808 that is formed of a round wire member 810 and that has a tissue manipulator first end 812, a tissue manipulator second end 814, a tissue manipulator length 815, a shaft 816, and a coil 818. The tissue manipulator length 815 extends from the tissue manipulator first end 812 to the tissue manipulator second end 814. A tissue manipulator can have any suitable length such as a tissue manipulator length equal to, greater than, less than, or about 3 inches, 4 inches, 5 inches, 6 inches, between about 3 inches and about 6 inches, about 4.724 inches, and any other length considered suitable for a particular embodiment. The tissue manipulator 808 can be used in combination with any of the medical devices described herein, such as medical device 10, medical device 110, medical device 410, and/or medical device 610. When included with a medical device, such as medical device 10, medical device 110, medical device 410, and/or medical device 610, the combined medical device and tissue manipulator 808 results in a tissue fixation system useful for fixing tissue in a desired position, as described in more detail herein.

The shaft 816 has a shaft lengthwise axis 817, a shaft first end 820, a shaft second end 822, and a shaft main body 824 that has a predefined shaft bend 826. The shaft lengthwise axis 817 extends through the shaft 816 from the shaft bend 826 to the shaft second end 822. The shaft main body 824 extends from the shaft first end 820 to the shaft second end 822 and has a first shaft main body portion 828, a second shaft main body portion 830, and a shaft outside diameter 825. The first shaft main body portion 828 extends from the shaft first end 820 to the shaft bend 826 and has a first shaft main body portion length 827. The second shaft main body portion 830 extends from the shaft bend 826 to the shaft second end 822 and has a second shaft main body portion length 829. In the illustrated embodiment, the first shaft main body portion length 827 is less than the second shaft main body portion length 829 and the shaft outside diameter 825 is continuous along the shaft main body 824. The first shaft main body portion length 827 is equal to about 2.5 centimeters and the second shaft main body portion length 829 is equal to about 12.7 centimeters.

The shaft bend 826 defines an angle 831 between the first shaft main body portion 828 and the second shaft main body portion 830. In the illustrated embodiment, the angle 831 is equal to about 90 degrees. This structural arrangement provides a mechanism for creating a crank that can be used to assist with introducing the tissue manipulator 808 into tissue, as described in more detail herein.

While the first shaft main body length 827 has been illustrated as being less than the second shaft main body length 829, the first shaft main body length and second shaft main body length of a shaft can be any suitable length and selection of a suitable length can be based on various considerations, such as the structural arrangement of the mounting plate, mounting jaw, and/or mounting clamp through which a tissue manipulator is intended to be passed and the structural arrangement and/or material that forms the portion of the body to which the tissue manipulator is intended to be attached. The inventors have determined that shafts having first shaft main body lengths that are between about 1.0 centimeter and about 5.0 centimeters are considered suitable. The inventors have determined that shafts having first shaft main body lengths that are between about 1.5 centimeters and about 4.0 centimeters are considered suitable. The inventors have determined that shafts having first shaft main body lengths that are between about 2.0 centimeters and about 3.0 centimeters are considered suitable. The inventors have determined that shafts having first shaft main body lengths that are about 2.5 centimeters are considered suitable. The inventors have determined that shafts having second shaft main body lengths that are between about 5.0 centimeters and about 20.0 centimeters are considered suitable. The inventors have determined that shafts having second shaft main body lengths that are between about 7.5 centimeters and about 15.0 centimeters are considered suitable. The inventors have determined that shafts having second shaft main body lengths that are between about 10.0 centimeters and about 13.0 centimeters are considered suitable. The inventors have determined that shafts having second shaft main body lengths that are about 12.0 centimeters are considered suitable. The inventors have determined that shafts having second shaft main body lengths that are about 12.7 centimeters are considered suitable.

The shaft outside diameter 827 can be any suitable outside diameter and selection of a suitable outside diameter can be based on various considerations, such as the structural arrangement of the mounting plate, mounting jaw, and/or mounting clamp through which a tissue manipulator is intended to be passed and the structural arrangement and/or material that forms the portion of the body to which the tissue manipulator is intended to be attached. The inventors have determined that shaft outside diameters that are between about 0.05 millimeters and about 1.0 millimeter are considered suitable. The inventors have determined that shaft outside diameters that are between about 0.20 millimeters and about 0.75 millimeters are considered suitable. The inventors have determined that shaft outside diameters that are between about 0.26 millimeters and about 0.5 millimeters are considered suitable. The inventors have determined that shaft outside diameters that are about 0.3 millimeters are considered suitable. The inventors have determined that shaft outside diameters that are about 0.5 millimeters are considered suitable. In the illustrated embodiment, the shaft outside diameter 827 is equal to about 0.5 millimeters. While the shaft outside diameter 827 has been illustrated as being continuous along the shaft main body 824, alternative embodiments can include a shaft main body that has a shaft outside diameter that varies along the shaft main body. In the illustrated embodiment, the shaft has sufficient column strength to maintain pressure or tension on tissue (e.g., a vocal cord, arytenoid muscle) and sufficient torsional rigidity to screw coil into tissue without undue flexing.

While the shaft bend 826 has been illustrated as defining an angle 831 between the first shaft main body portion 828 and the second shaft main body portion 830 that is equal to about 90 degrees, a shaft bend included on a tissue manipulator can define any suitable angle between a first shaft main body portion and a second shaft main body portion. Selection of a suitable angle to define between a first shaft main body portion and a second shaft main body portion can be based on various considerations, such as the structural arrangement of a medical device through which a tissue manipulator is intended to be passed and/or the structural arrangement of the portion of the body to which a tissue manipulator is intended to be attached. Examples of suitable angles for a shaft bend to define between a first shaft main body portion and a second shaft main body portion include angles equal to, about, less than, or greater than, 90 degrees, 45 degrees, and any other angle considered suitable for a particular embodiment.

While the shaft bend 826 has been illustrated as being a predefined bend in the shaft main body 824, alternative embodiments can omit the inclusion of a predefined shaft bend such that the shaft main body defines an elongate shaft that has a continuous, uninterrupted, main body. In embodiments that omit the inclusion of a shaft bend and a shaft bend is desired, a shaft bend can be created by a user of a tissue manipulator prior to performing, or during, treatment by applying a first force on the first shaft main body portion and maintaining the position of, or applying a second force on, the second shaft main body portion to create the shaft bend, or vice versa. If both the first and second forces are applied on the shaft, each of the first force and the second force is directed toward a plane that extends through the shaft lengthwise axis.

The coil 818 is disposed between the tissue manipulator first end 812 and the tissue manipulator second end 814 and is attached to the shaft 816 at the shaft second end 822. The coil 818 has a coil lengthwise axis 819, a coil first end 836, a coil second end 838 that defines a sharp tip, and a coil main body 840 that extends from the coil first end 836 to the coil second end 838. The coil main body 840 has a coil main body length 837, a first coil main body outside diameter 839, a second coil main body outside diameter 841, and defines a plurality of coil turns 842 creating a helical coil 844. The coil lengthwise axis 819 is coaxial with the shaft lengthwise axis 817 and extends through the plurality of coil turns 842. The coil main body length 837 extends from the coil first end 836 to the coil second end 838 and is measured along the coil lengthwise axis 819. In the illustrated embodiment, the plurality of coil turns 842 comprises four complete coil turns that extend clockwise about the coil lengthwise axis 819 creating the helical coil 844. In the illustrated embodiment, the plurality of coil turns 842 has a maximum coil turn outside diameter 843, as illustrated in FIG. 35, that is equal to about three times the shaft outside diameter 827. In the embodiment illustrated, the maximum coil turn outside diameter 843 is equal to about 1.5 millimeters and the helical coil 844 is a 45 degree helical coil and defines a space 846 between adjacent turns of the coil 818 that has a length 847 equal to about 0.015 inches. Alternative embodiments, however, can have any suitable coil length (e.g., between about 0.10 inches and about 0.175 inches, about 0.1647 inches) or maximum coil outside diameter (e.g., between about 1 millimeter and about 1.524 millimeters, about 1.143 millimeters).

The first coil main body outside diameter 839 is greater than the second coil main body outside diameter 841 and the outside diameter of the coil main body 840 tapers from the coil first end 836 to the coil second end 838. The coil outside diameter can taper at any suitable rate along the coil main body length 837 and the first coil main body outside diameter 839 and second coil main body outside diameter 841 can be any suitable outside diameter. Selection of a suitable coil main body outside diameter can be based on various considerations, such as the structural arrangement of the mounting plate, mounting jaw, and/or mounting clamp through which a tissue manipulator is intended to be passed and the structural arrangement and/or material that forms the portion of the body to which the tissue manipulator is intended to be attached (e.g., thyroid cartilage). In the illustrated embodiment, the first coil main body outside diameter 839 is equal to the shaft outside diameter 825. However, alternative embodiments can include a coil that has a first coil main body outside diameter that is equal to, about, less than, or greater than the shaft outside diameter. The inventors have determined that second coil main body outside diameters that are between about 0.06 millimeters and about 0.9 millimeters are considered suitable. The inventors have determined that second coil main body outside diameters that are between about 0.10 millimeters and about 0.60 millimeters are considered suitable. The inventors have determined that second coil main body outside diameters that are between about 0.12 millimeters and about 0.24 millimeters are considered suitable. The inventors have determined that second coil main body outside diameters that are about 0.12 millimeters are considered suitable. The inventors have determined that second coil main body outside diameters that are about 0.24 millimeters are considered suitable. In the illustrated embodiment, the second coil main body outside diameter 835 is equal to about 0.24 millimeters.

While the coil 818 has been illustrated as having a coil lengthwise axis 819 that is coaxial with the shaft lengthwise axis 817, a plurality of coil turns 842 in a clockwise direction, a maximum coil turn outside diameter 843, and defining a 45 degree helical coil 844 that defines a 0.015 space 846 between adjacent turns of the coil 818, a coil can have any suitable structural arrangement. Selection of a suitable structural arrangement for a coil can be based on various considerations, such as the structural arrangement and/or material that forms the portion of the body to which a tissue manipulator is intended to be attached. For example, alternative embodiments can include a coil lengthwise axis that is not coaxial with a shaft lengthwise axis (e.g., a coil lengthwise axis is parallel with, or disposed at an angle to, a shaft lengthwise axis) and/or a coil that defines counterclockwise turns. Examples number of turns considered suitable to include on coil include a partial turn, one complete turn, at least one complete turn, two complete turns, three complete turns, four complete turns, five complete turns, six complete turns, seven complete turns, eight complete turns, nine complete turns, ten complete turns, more than ten complete turns, between four and eight complete turns, more than eight complete turns, and any other number considered suitable for a particular embodiment. Examples of maximum coil turn outside diameters considered suitable for a coil include diameters equal to, about, less than, or greater than, one time, two times, three times, four times, or five times the shaft outside diameter of a tissue manipulator. Examples of types of coils that can be created by the coil main body of a coil by include helical coils, conical coils, 45 degree helical coils, 45 degree conical coils, coils that have a rectangular, square, round, or elongated, cross-sectional shape, coils that have any suitable spacing between adjacent turns, coils that have any suitable angular arrangement, single helical coils, double helical coils, and any other coil considered suitable for a particular embodiment. For example, as illustrated in FIG. 35A, a tissue manipulator 808′ can include a first shaft 816′, a second shaft 816″, a first coil 818′, and a second coil 818″. Each of the first shaft 816′ and the second shaft 816″ can include structure similar to the shaft described herein. Each of the first coil 816′ and the second coil 816″ can include structure similar to the coils described herein.

While the outside diameter of the coil main body 840 has been illustrated as tapering from the coil first end 836 to the coil second end 838, the coil main body of a coil can taper along any suitable portion of a coil main body length. Selection of a suitable main body length to include a tapering diameter can be based on various considerations, such as the structural arrangement and/or material that forms the portion of the body to which a tissue manipulator is intended to be attached. For example, the outside diameter of a coil main body can taper from a first location disposed between the coil first end and the coil second end to the second end, or a second location disposed between the first location and the coil second end.

The tissue manipulator 808 can be formed of any suitable material and selection of a suitable material to form a tissue manipulator according to a particular embodiment can be based on various considerations, including the material forming a tissue fixation device and/or the portion of the body to which a tissue manipulator is intended to be attached. Example materials considered suitable to form a tissue manipulator include rigid materials, semi-rigid materials, implant grade materials, implant grade rigid materials, implant grade semi-rigid materials, biocompatible materials, materials that can be made biocompatible, metals such as stainless steel, titanium, cobalt chromium, metal alloys, titanium alloys, cobalt chromium alloys, nickel-titanium alloy (e.g., Nitinol), thermoplastics, polymers, polyetheretherketone (PEEK), Pebax, nylon, polyethylene, high-density polyethylene (HDPE), high-performance polyethylene (HPPE), polyurethane, silicone, acrylonitrile butadiene styrene (ABS), polyoxymethylene (e.g., acetal), materials that have a round cross-sectional configuration, materials that have a rectangular, elongated, or square, cross-sectional configuration, materials that have a first cross-sectional configuration along a first portion of its length (e.g., round) and a second cross-sectional configuration along a second portion of its length (e.g., rectangular), combinations of those described herein, and any other material considered suitable for a particular application. In the illustrated embodiment, the tissue manipulator 808 is formed of an implant-grade metal alloy. Alternative embodiments, however, can include a tissue manipulator that is formed of a material that is the same as, or different than, a material that forms a mounting plate, mounting jaw, mounting clamp, and/or a fastener of a plurality of fasteners. For example, if a tissue manipulator is formed of a material that is relatively more flexible than the material that forms a mounting plate, mounting jaw, and/or mounting clamp (e.g., the tissue manipulator is formed of a material that has a durometer hardness that is less than the durometer hardness of the material that forms a mounting plate, mounting jaw, and/or mounting clamp) the material that forms the tissue manipulator can flex, or deform, when the tissue manipulator is pinched between the mounting plate and mounting jaw and/or pinched between the mounting plate and mounting clamp.

FIG. 36 illustrates another example tissue manipulator 908 for the fixation of tissue. The tissue manipulator 908 is similar to the tissue manipulator 808 illustrated in FIGS. 34 and 35 and described above, except as detailed below. The tissue manipulator 908 comprises a tissue manipulator first end 912, a tissue manipulator second end 914, a shaft 916, and a coil 918. The tissue manipulator 908 can be used in combination with any of the medical devices described herein, such as medical device 10, medical device 110, medical device 410, and/or medical device 610. When included with a medical device, such as medical device 10, medical device 110, medical device 410, and/or medical device 610, the combined medical device and tissue manipulator 908 result in a tissue fixation system useful for fixing tissue in a desired position, as described in more detail herein

In the illustrated embodiment, the tissue manipulator 908 defines a shoulder 932 at the location where the shaft 916 meets the coil 918, the shaft 916 has a shaft outside diameter 925 that is constant from the shaft first end 920 to the shaft second end 922, the coil 918 has a coil main body outside diameter 938 that is constant from the coil first end 936 to the coil second end 938, and the plurality of coil turns 942 comprises three complete turns about the coil lengthwise axis 919. In addition, the coil lengthwise axis 919 is offset, not coaxial with, the shaft lengthwise axis 917.

The structural arrangement of the tissue manipulator 908 illustrated in FIG. 36, or in any other embodiment, can be accomplished by forming the manipulator from a unitary piece of material or forming a shaft of a first piece of material and attaching it to a second piece of material that forms a coil. Attachment between a first piece of material and a second piece of material can be accomplished using any suitable method of attachment, such as welding, fusing, using adhesive, and any other technique or method considered suitable for a particular embodiment.

FIG. 37 illustrates another example tissue manipulator 1008 for the fixation of tissue. The tissue manipulator 1008 is similar to the tissue manipulator 808 illustrated in FIGS. 34 and 35 and described above, except as detailed below. The tissue manipulator 1008 comprises a tissue manipulator first end 1012, a tissue manipulator second end 1014, a shaft 1016, and a coil 1018. The tissue manipulator 1008 can be used in combination with any of the medical devices described herein, such as medical device 10, medical device 110, medical device 410, and/or medical device 610. When included with a medical device, such as medical device 10, medical device 110, medical device 410, and/or medical device 610, the combined medical device and tissue manipulator 1008 result in a tissue fixation device useful for fixing tissue in a desired position, as described in more detail herein.

In the illustrated embodiment, the coil 1018 has a plurality of coil turns 1042 that comprises four complete turns and creates a conical coil 1044 that has a first coil main body outside diameter 1039 and a second coil main body outside diameter 1041 that is less than the first main body outside diameter. In the illustrated embodiment, the coil outside diameter tapers from the coil first end 1036 to the coil second end 1038 and the coil main body outside diameter tapers from the coil first end 1036 to the coil second end 1038.

Various methods of manufacturing a tissue manipulator are described herein. While the methods described herein are shown and described as a series of acts, it is to be understood and appreciated that the methods are not limited by the order of acts, as some acts may in accordance with these methods, occur in different orders, and/or concurrently with other acts described herein.

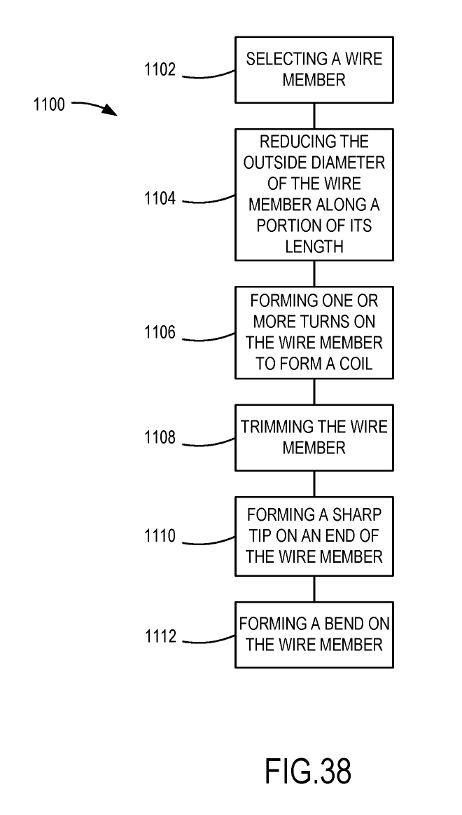

FIG. 38 is a schematic illustration of a method 1100 of manufacturing a tissue manipulator.

An initial step 1102 comprises selecting a wire member to form a tissue manipulator. Another step 1104 comprises reducing the outside diameter of the wire member along a portion of its length. Another step 1106 comprises forming one or more turns along a portion of the length of the wire member to form a coil. Another step 1108 comprises trimming the wire member to a desired length. Another step 1110 comprises forming a sharp tip on an end of the wire member. Another step 1112 comprises forming a bend on the wire member.

Step 1102 can be accomplished by selecting any suitable wire member and selection of a suitable wire member can be based on various considerations, such as the structural arrangement of a medical device through which a tissue manipulator is intended to be passed. For example, a round wire member that has a length at least, greater than, or equal to, about 12.7 centimeters long and an outside diameter between about 0.254 millimeters and about 0.5 millimeters are considered suitable. However, as described herein, use of other wire member sizes and configurations are considered suitable.

Step 1104 can be accomplished using any suitable technique or method of reducing the outside diameter, or tapering the outside diameter, of a portion of the tissue manipulator length that extends from a location between the tissue manipulator first end and the tissue manipulator second end to the tissue manipulator second end. Selection of a suitable technique or method of reducing, or tapering, the outside diameter of a portion of the tissue manipulator length can be based on various considerations, such as the material(s) that forms the tissue manipulator. Examples of suitable techniques and methods for reducing, or tapering, the outside diameter of a portion of the tissue manipulator length include center-less grinding, electrolysis, acid-dipping, pulling the portion of the tissue manipulator length under tension, and any other suitable technique or method considered suitable for a particular embodiment.

Step 1106 can be accomplished using any suitable technique or method of forming one or more turns along a portion of the length of a wire member to form a coil and selection of a suitable technique or method can be based on various considerations, such as the material(s) that forms the wire member. Examples of suitable techniques and methods of forming one or more turns along a portion of the length of a wire member to form a coil include using a mandrel, using a form, and any other suitable technique or method considered suitable for a particular embodiment. For example, the portion of the wire member intended to form a coil (e.g., the portion having a reduced or tapered outside diameter) is pulled around an approximately 0.010 inch diameter mandrel to produce a coil having a 0.010 inch inside diameter. While a mandrel having a particular outside diameter has been described, a mandrel used to create a coil on a wire member can have any suitable outside diameter and selection of a suitable outside diameter for a mandrel can be based on various considerations, such as the intended use of the tissue manipulator being manufactured.

An optional step comprises adjusting the coil lengthwise axis to ensure that it is positioned relative to the shaft lengthwise axis as desired (e.g., coaxial, parallel to, at an angle to). Another optional step comprises adjusting the coil to ensure that it has the desired structural configuration (e.g., distance between turns, angle of turns). These optional steps can be accomplished by maintaining the position of the shaft and manipulating the position of the coil by applying an appropriate force on the coil (e.g., axial, torque), maintaining the position of the coil and manipulating the position of the shaft by applying an appropriate force on the shaft (e.g., axial, torque), or manipulating the position of the shaft while concurrently manipulating the position of the coil by applying an appropriate force on the shaft and coil (e.g., axial, torque).

Step 1108 can be accomplished using any suitable technique or method of trimming a wire member and selection of a suitable technique or method can be based on various considerations, such as the material(s) that forms the wire member. Examples of suitable techniques and methods of trimming a wire member include cutting using a cutting tool (e.g., snips), grinding, and any other suitable technique or method considered suitable for a particular embodiment. For example, the shaft length and number of coil turns included on a tissue manipulator can be based on the anatomy of a patient (e.g., thickness of the arytenoid muscle, distance between thyroid cartilage and arytenoid muscle) and the number of coil turn on a tissue manipulator and/or the shaft length of a tissue manipulator can be trimmed based on the anatomy of the patient. Optional steps include trimming the shaft length, trimming the coil length, trimming the number of coil turns. Example lengths considered suitable to trim a shaft length and/or coil length are described herein.

Step 1110 can be accomplished using any suitable technique or method of creating a sharp tip on a tissue manipulator second end and selection of a suitable technique or method can be based on various considerations, such as the material(s) that forms the wire member. Examples of suitable techniques and methods of creating a sharp tip on a wire member include grinding, filing, electrolysis, and any other suitable technique or method considered suitable for a particular embodiment.

Step 1112 can be accomplished using any suitable technique or method of forming a bend on the wire member and selection of a suitable technique or method can be based on various considerations, such as the material(s) that forms the wire member. For example, step 1112 can be accomplished by maintaining the position of a first portion of the shaft that extends from the shaft first end toward the shaft second end and manipulating the position of a second portion of the shaft that extends from the shaft second end toward the shaft first end by applying an appropriate force on the second portion (e.g., torque), manipulating the position of a first portion of the shaft that extends from the shaft first end toward the shaft second end and maintaining the position of a second portion of the shaft that extends from the shaft second end toward the shaft first end by applying an appropriate force on the first portion (e.g., torque), or manipulating the position of a first portion of the shaft that extends from the shaft first end toward the shaft second end and manipulating the position of a second portion of the shaft that extends from the shaft second end toward the shaft first end by applying an appropriate force on the first portion and second portion (e.g., torque).

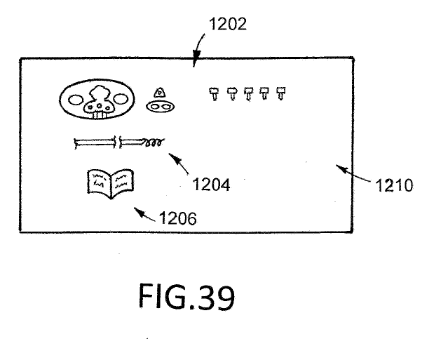

FIG. 39 illustrates an exemplary kit 1210 comprising a medical device 1202 according to an embodiment, such as medical device 610 illustrated in FIGS. 24, 25, 26, 27, 28, 29, 30, 31, 32, and 33; a tissue manipulator 1204 according to an embodiment, such as tissue manipulator 1008 illustrated in FIG. 37; and instructions for use 1206.

While kit 1210 has been illustrated as only including a single medical device 1202 and a single tissue manipulator 1204, any suitable number, and type, of medical devices and/or tissue manipulators can be included in a kit. Selection of a suitable number of medical devices and tissue manipulators to include in a kit according to a particular embodiment can be based on various considerations, such as the treatment intended to be performed. Examples of suitable numbers of medical devices and/or tissue manipulators to include in a kit include at least one, one, two, a plurality, three, four and any other number considered suitable for a particular embodiment.

Furthermore, while medical device 610 and tissue manipulator 1008 have been illustrated as included in kit 1202, any suitable medical device and any suitable tissue manipulator can be included in a kit. Selection of a suitable medical device and tissue manipulator to include in a kit according to a particular embodiment can be based on various considerations, such as the treatment intended to be performed. Examples of medical devices considered suitable to include in a kit include medical device 10, medical device 110, medical device 410, medical device 610, variations of the medical devices described herein, such as those that include an alternative mounting plate (e.g., mounting plate 312), and/or any other medical device considered suitable for a particular embodiment. Examples of tissue manipulators considered suitable to include a kit include tissue manipulator 808, tissue manipulator 808′, tissue manipulator 908, tissue manipulator 1008, variations of the tissue manipulators described herein, and/or any other tissue manipulator considered suitable for a particular embodiment.

In an alternative embodiment, a kit can include a first medical device, a second medical device, a third medical device, and a tissue manipulator. The first, second, and third medical devices can be the substantially the same structurally, or different from one another. The second medical device and third medical device can be scaled relative to the first medical device. A kit that includes one or more medical devices that are scaled relative to one another provides a mechanism for selecting a medical device that best fits a particular patient's anatomy. For example, the first medical device can be sized and configured for a pediatric patient, the second medical device can be 2:1 scale of the first medical device and be sized and configured for teenage patient, and the third medical device can be 4:1 scale of the first medical device and be sized and configured for an adult patient.

In embodiments in which more than one medical device is included in a kit, a second medical device and/or third medical device can be scaled relative to a first medical device based on any suitable proportional ratio. Selection of a suitable amount to scale a second medical device and/or third medical device relative to a first medical device can be based on various considerations, including the treatment intended to be performed. For example, a second medical device and/or third medical device can be scaled such that it is 1:1, 2:1, 3:1, 4:1, 5:1, 6:1, 7:1 relative to a first medical device, between about 1:1 and about 7:1 relative to a first medical device, and any other proportional ratio considered suitable for a particular embodiment.

Various methods of treatment are described herein. While the methods described herein are shown and described as a series of acts, it is to be understood and appreciated that the methods are not limited by the order of acts, as some acts may in accordance with these methods, occur in different orders, and/or concurrently with other acts described herein.

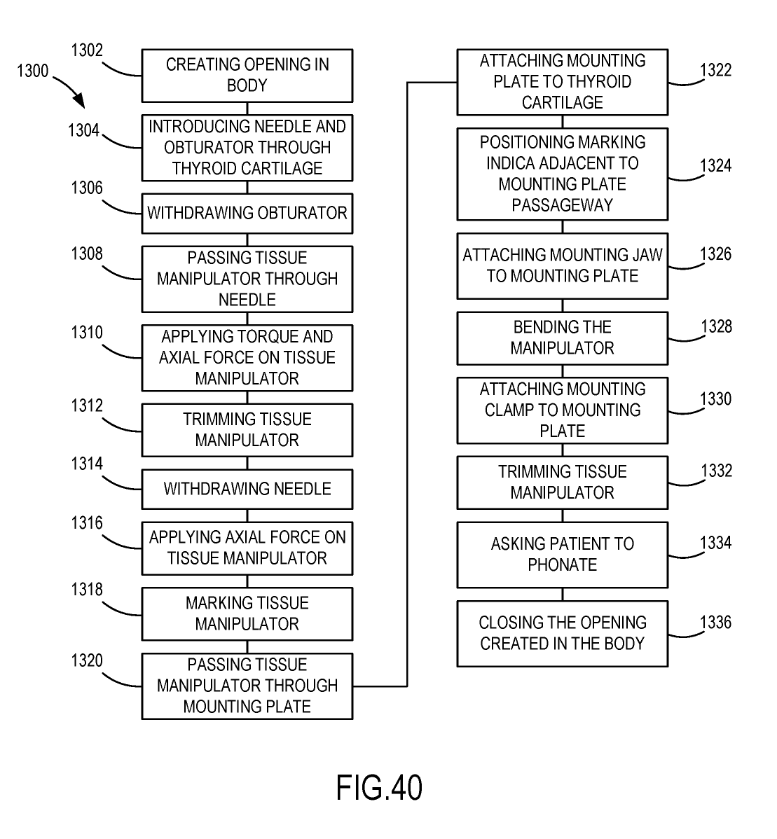

FIG. 40 is a schematic illustration of an example method of treatment 1300 using a tissue fixation system that includes an example medical device and an example tissue manipulator. In the example described herein, the method of treatment comprises a method of repositioning an arytenoid muscle resulting in repositioning of a vocal cord.

A step 1302 comprises creating an opening in the body over the vocal cord intended to be treated to access the thyroid cartilage. Another step 1304 comprises introducing a needle and an obturator through the thyroid cartilage and into the arytenoid muscle. Another step 1306 comprises withdrawing the obturator while maintaining position of needle. Another step 1308 comprises passing a tissue manipulator through the needle until it contacts the arytenoid muscle. Another step 1310 comprises applying torque to the tissue manipulator while applying an axial force toward the arytenoid muscle such that the tissue manipulator rotates and engages the arytenoid muscle. Another step 1312 comprises trimming the tissue manipulator between the shaft bend and the shaft second end to create a new tissue manipulator end. Another step 1314 comprises withdrawing the needle from the tissue and thyroid cartilage while maintaining position of the tissue manipulator. Another step 1316 comprises applying an axial force on the tissue manipulator to achieve a desired placement of vocal cord. Another step 1318 comprises marking the shaft of the tissue manipulator where it exits the thyroid cartilage while holding the tissue manipulator at desired location. Another step 1320 comprises passing the new tissue manipulator end through the mounting plate passageway of a mounting plate. Another step 1322 comprises attaching the mounting plate to the thyroid cartilage. Another step 1324 comprises positioning the marking indicia on the tissue manipulator adjacent to the mounting plate passageway. Another step 1326 comprises attaching the mounting jaw to the mounting plate such that a portion of the tissue manipulator is disposed between the mounting plate and the mounting jaw. Another step 1328 comprises bending the portion of the tissue manipulator extending from the mounting plate at the marking indicia on the tissue manipulator toward the midline of the throat. Another step 1330 comprises attaching the mounting clamp to the mounting plate such that a portion of the tissue manipulator is disposed between the mounting plate and the mounting clamp. Another step 1332 comprises trimming tissue manipulator. Another step 1334 comprises asking the patient to phonate to evaluate the position of the vocal cord and to determine whether adjustments are needed. Another step 1336 comprises closing the opening made in the body.

Step 1302 can be accomplished can be accomplished using any suitable medical device (e.g., scalpel) and by creating the opening on the side of the vocal cord that is being treated and over the thyroid cartilage. Step 1302 can be accomplished by creating an opening that is sized and configured to allow a medical device and/or tissue manipulator to pass through the opening.

An optional step comprises administering medication near a point of treatment. This optional step can be accomplished prior to step 1302 by administering any suitable medication at any suitable location on the body of a patient. For example, medication can be administered on the side of the vocal cord being treated and over the thyroid cartilage. Any suitable medication such as a local anesthetic, combination of local anesthetic and vasoconstrictor (e.g., 1% lidocaine, with 1:100,000 epinephrine), and/or general anesthetic can be used and administered in any suitable manner, such as subcutaneously using a convention syringe.

Another optional step that can be completed prior to step 1302 comprises asking the patient to phonate. This optional step can be accomplished by providing instructions to a patient to phonate. Another optional step that can be completed prior to step 1302, or subsequent to step 1302, comprises determining how a vocal cord needs to be modified for effective treatment based on patient's anatomy and/or phonation. This step can be accomplished by visualizing the vocal cord using a scope that is partially disposed in a patient's larynx and/or by listening to the patient phonate.

Another optional step comprises dissecting the underlying soft tissues to expose the thyroid cartilage. This optional step can be accomplished subsequent to step 1302 using any suitable medical device such that the thyroid cartilage is exposed. For example, this optional step can be accomplished using one or more of a needle, probe, pick, scalpel, forceps, tweezers, scissors, or any other device considered suitable for a particular procedure.

Another optional step that can be completed subsequent to step 1302 comprises creating one or more pilot holes through the thyroid cartilage sized and configured to receive a tissue manipulator and/or a fastener of the plurality of fasteners of a medical device. This optional step can be accomplished using any suitable drill and a drill bit that has a first outside diameter. A drill bit used complete any step of the methods described herein can have any suitable outside diameter, and skilled artisans will be able to select a suitable outside diameter for a drill bit according to a particular embodiment based on various considerations, including the structural arrangement at a point of treatment. Example outside diameters considered suitable for a drill bit include outside diameters that are less than, or equal to, or about the minor diameter and/or the major diameter of a fastener being passed through a pilot hole.

Step 1304 can be accomplished using any needle and obturator considered suitable for a particular embodiment and selection of a suitable needle and obturator can be based on various considerations, such as the structural configuration of a medical device and/or tissue manipulator intended to be implanted. For example, needles that are 20 gage or smaller are considered suitable. The needle comprises a needle first end and a sharp needle second end and defines a lumen that extends from the needle first end to the needle second end. The obturator comprises an obturator first end and a sharp obturator second end and may optionally contain a light source. Alternatively, the obturator second end can be rounded or blunt. The obturator is disposed within the needle lumen.

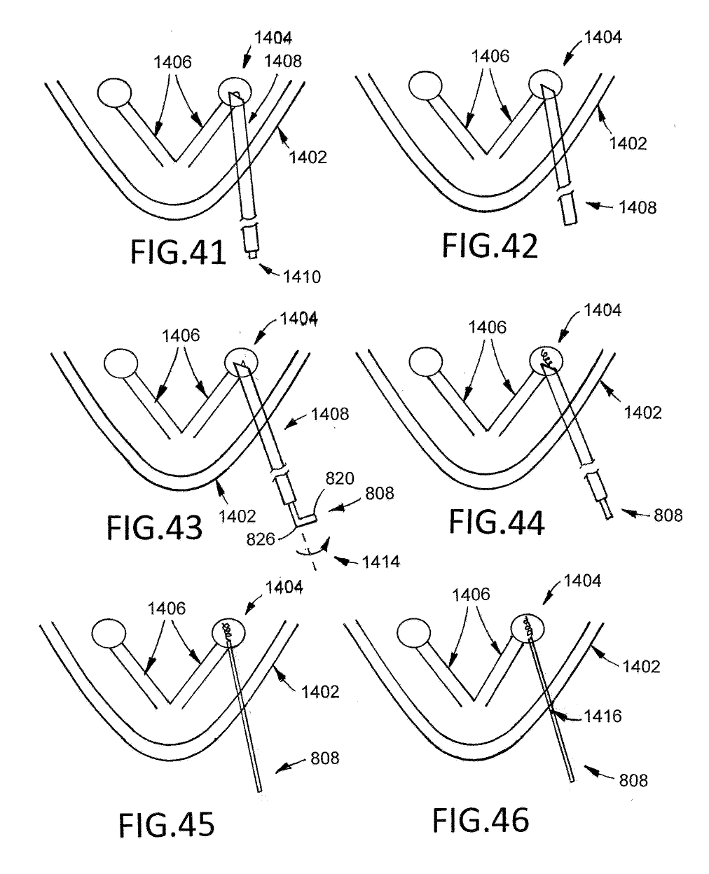

Step 1304 can be accomplished by locating the thyroid cartilage, positioning the needle second end and the obturator second end on the thyroid cartilage, and applying an axial force toward the thyroid cartilage on the needle and obturator such that it passes through the thyroid cartilage and into the arytenoid muscle (e.g., translaryngeal insertion of needle and obturator). It is considered advantageous to introduce the needle and obturator into the arytenoid muscle from the anterior to the posterior and to stop the application of the axial force on the needle and obturator before the needle second end and/or obturator second end penetrates the posterior surface of the arytenoid muscle. FIG. 41 illustrates a superior view of the thyroid cartilage 1402, the arytenoid muscle 1404, and vocal cords 1406 with the needle 1408 and obturator 1410 disposed through the thyroid cartilage 1402 and partially disposed within the arytenoid muscle 1404. Optionally, step 1304 can be accomplished using a laryngoscope disposed within a portion of the larynx to facilitate direct visualization of the needle and/or obturator. An optional step comprises confirming the placement of the needle second end and/or obturator second end using the laryngoscope and/or transillumination.

Step 1306 can be accomplished by maintaining the position of the needle and applying an axial force on the obturator away from the thyroid cartilage until the obturator becomes free of the needle lumen. FIG. 42 illustrates the needle 1408 disposed through the thyroid cartilage 1402 and partially disposed within the arytenoid muscle 1404 and the obturator 1410 withdrawn from the needle lumen.

Step 1308 can be accomplished by introducing the tissue manipulator second end into the needle lumen and applying an axial force on the tissue manipulator toward the thyroid cartilage until the tissue manipulator second end contacts the arytenoid muscle (e.g., inside the rearmost end of the arytenoid muscle). Step 1308 can be accomplished by applying an axial force on any suitable portion (e.g., shaft) of any suitable tissue manipulator according to an embodiment, such as the embodiments described herein. Selection of a suitable tissue manipulator to pass through a needle lumen can be based on various considerations, including the structural arrangement of the arytenoid muscle being treated. Example tissue manipulators considered suitable include tissue manipulator 808, tissue manipulator 808′, tissue manipulator 908, tissue manipulator 1008, and any other tissue manipulator considered suitable for a particular embodiment. FIG. 43 illustrates a superior view of the thyroid cartilage 1402 and the arytenoid muscle 1404 with the needle 1408 and tissue manipulator 808 disposed through the thyroid cartilage 1402 and partially disposed within the arytenoid muscle 1404. In the method of treatment 1300 described herein, the tissue manipulator 808 illustrated and described with respect to FIGS. 34 and 35 has been described as being releasably attached to the arytenoid muscle and a medical device. Optionally, a tissue manipulator can include a marking indicia disposed along the shaft length that is positioned at a location from the tissue manipulator second end that is equal to, or about, the length of the needle such that when the coil second end is disposed adjacent to the needle second end the marking indicia is disposed adjacent to the needle first end.

Step 1310 can be accomplished by applying torque to any suitable portion (e.g., shaft, first shaft main body portion, crank) of the tissue manipulator while applying an axial force toward the arytenoid muscle such that the tissue manipulator rotates and engages the arytenoid muscle (e.g., sufficient purchase in the arytenoid is achieved). Depending on the direction in which the turns of the coil of the tissue manipulator are configured, torque can be applied to the tissue manipulator in either a clockwise or counterclockwise direction about the shaft lengthwise axis of the tissue manipulator. Arrow 1414 illustrated in FIG. 43 shows the application of torque on the tissue manipulator 808 in a clockwise direction. This step is completed until approximately 3 or 4 turns of the tissue manipulator 808 are engaged with the tissue and such that the tissue manipulator does not extend through the posterior surface of the arytenoid muscle.

Step 1312 can be accomplished using any suitable technique or method of trimming a tissue manipulator and selection of a suitable technique or method can be based on various considerations, such as the material(s) that forms the tissue manipulator. Examples of suitable techniques and methods of trimming a tissue manipulator include cutting using a cutting tool (e.g., snips), grinding, and any other suitable technique or method considered suitable for a particular embodiment. It is considered advantageous to trim the tissue manipulator between the shaft bend and the shaft second end to allow a mounting plate to be easily passed over the tissue manipulator, as described in more detail herein. Alternative embodiments, however, can include a step that comprises trimming the tissue manipulator between the shaft bend and the shaft first end, trimming the tissue manipulator at the shaft bend, or omitting step 1312. FIG. 44 illustrates the tissue manipulator 808 trimmed between the shaft bend and the shaft second end.

Step 1314 can be accomplished by maintaining the position of the tissue manipulator and applying an axial force away from the thyroid cartilage on the needle until the needle is free of the thyroid cartilage. FIG. 45 illustrates the tissue manipulator 808 disposed through the thyroid cartilage 1402 and partially disposed within the arytenoid muscle 1404 and the needle withdrawn from the thyroid cartilage 1402.

Step 1316 can be accomplished by applying an axial force toward, or away from, the thyroid cartilage on any suitable portion of the shaft of the tissue manipulator to position the arytenoid muscle in a desired location that can be based on the patient's anatomy or that results in a desired phonation. Alternatively, torque can be applied separately, or in combination with the axial force, to the shaft of the tissue manipulator (e.g., clockwise or counterclockwise direction about the shaft lengthwise axis) to achieved desired placement of vocal cord. FIG. 46 illustrates the tissue manipulator 808 disposed through the thyroid cartilage 1402 and partially disposed within the arytenoid muscle 1404. The arytenoid muscle 1404 has been positioned in a desired location such that the vocal cord 1406 achieves the desired phonation.

An optional step that can be completed prior to and/or subsequent to step 1316 comprises asking the patient to phonate. This optional step can be accomplished by providing instructions to a patient to phonate. Another optional step that can be completed subsequent to step 1316 comprises adjusting the position of the tissue manipulator. This optional step can be accomplished by applying an axial force toward, or away from, the thyroid cartilage on any suitable portion of the shaft of the tissue manipulator to position the arytenoid muscle in a desired location that can be based on the patient's anatomy or that results in a desired phonation. Alternatively, torque can be applied separately, or in combination with the axial force, to the shaft of the tissue manipulator (e.g., clockwise or counterclockwise direction about the shaft lengthwise axis) to achieved desired placement of vocal cord.

Step 1318 can be accomplished using any suitable marking implement such as writing implements, pens, markers, and any other marking implement considered suitable for a particular embodiment. FIG. 46 illustrates a marking indicia 1416 disposed on the tissue manipulator 808. Optionally, step 1318 can be omitted from method 1300.

Step 1320 can be accomplished by positioning the mounting plate back surface toward the thyroid cartilage and applying an axial force on the mounting plate toward the thyroid cartilage such that the new tissue manipulator end passes through the mounting plate passageway. Step 1320 can be accomplished by applying an axial force on any suitable portion (e.g., mounting plate main body) of the mounting plate. Any suitable medical device according to an embodiment can be used to complete the methods described herein, such as the embodiments described and illustrated herein. Selection of a suitable medical device can be based on various considerations, including the structural arrangement of the thyroid cartilage to which a medical device is intended to be attached. Example medical devices considered suitable include medical device 10, medical device 110, medical device 410, medical device 610, and any other medical device considered suitable for a particular embodiment.

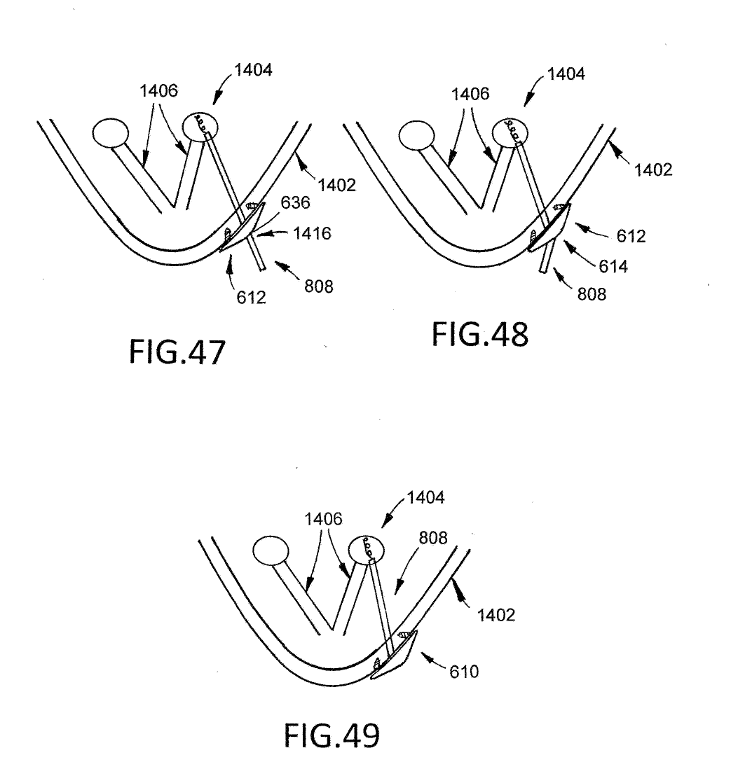

Step 1322 can be accomplished by passing a fastener through the mounting plate main body and into the thyroid cartilage. FIG. 47 illustrates the mounting plate 612 of the medical device 610 illustrated in FIG. 27 attached to the thyroid cartilage 1402 and the tissue manipulator disposed through the mounting plate passageway 636 and partially disposed within the arytenoid muscle 1404. While method 1300 has been illustrated as being accomplished such that a tissue manipulator has been attached to the arytenoid muscle and a mounting plate is attached to the thyroid cartilage, a tissue manipulator can be attached to any muscle of a larynx system that will provide proper treatment of a vocal cord and a mounting plate can be attached to any suitable portion of a body that provides fixation of a tissue manipulator and access to the desired anatomy of a patient.

Step 1324 can be accomplished by applying an axial force on the shaft of the tissue manipulator toward, or away from, the thyroid cartilage until the marking indicia on the tissue manipulator is disposed adjacent to the mounting plate passageway (e.g., the mark is flush with the mounting plate passageway at the mounting plate top surface). FIG. 47 illustrates the marking indicia 1416 disposed on the tissue manipulator 808 and adjacent to the mounting plate passageway 636. Alternatively, in embodiments in which a mark has not been applied to the tissue manipulator, step 1324 can alternatively comprise positioning the tissue manipulator adjacent to the mounting plate passageway while asking a patient to phonate to confirm that the position of the tissue manipulator results in a desired phonation from the patient.

An optional step that can be completed prior to and/or subsequent to step 1324 comprises asking the patient to phonate while the marking indicia is disposed adjacent to the mounting plate passageway to confirm proper placement of the tissue manipulator, arytenoid muscle, and vocal cord. This optional step can be accomplished by providing instructions to a patient to phonate. Another optional step comprises marking the tissue manipulator in a second location based on the results of the optional step of asking the patient the phonate.

Step 1326 can be accomplished by passing a first fastener through the mounting jaw aperture and into the mounting plate first aperture. FIG. 48 illustrates the mounting jaw 614 attached to the mounting plate 612. This step advantageously releasably attaches the tissue manipulator between the mounting plate and the mounting jaw and creates a first pinch point along the length of the tissue manipulator between the mounting plate and the mounting jaw. Alternatively, step 1326 can comprise attaching the mounting jaw to mounting plate while the marking indicia is positioned adjacent the mounting plate passageway. Alternatively, step 1326 can comprise partially attaching the mounting jaw to mounting plate. Another optional step comprises completely attaching the mounting jaw to the mounting plate while maintaining the position of the tissue manipulator.

An optional step that can be completed prior to and/or subsequent to step 1326 comprises asking the patient to phonate to determine whether position of the vocal cord is satisfactory. This optional step can be accomplished by providing instructions to a patient to phonate and listening to the patient's phonation. Another optional step that can be completed subsequent to step 1326 comprises adjusting the position of the tissue manipulator. This optional step can be accomplished by applying an axial force toward, or away from, the thyroid cartilage on any suitable portion of the shaft of the tissue manipulator to position the arytenoid muscle in a desired location that can be based on the patient's anatomy or that results in a desired phonation. Alternatively, torque can be applied separately, or in combination with the axial force, to the shaft of the tissue manipulator (e.g., clockwise or counterclockwise direction about the shaft lengthwise axis) to achieved desired placement of vocal cord.

Step 1328 can be accomplished by applying an axial force on the shaft of the tissue manipulator orthogonal to the shaft lengthwise axis and toward the midline of the throat such that a bend is formed along the shaft between the new tissue manipulator end and the shaft second end. FIG. 48 illustrates the tissue manipulator 808 bent toward the midline of the throat.

Step 1330 can be accomplished by passing a second fastener through the mounting clamp first aperture and the mounting plate second aperture and a third fastener through the mounting clamp second aperture and the mounting plate third aperture. This step advantageously releasably attaches the tissue manipulator to between the mounting plate and the mounting clamp and creates a second pinch point along the length of the tissue manipulator between the mounting plate and the mounting clamp. FIG. 49 illustrates mounting plate 616 and mounting jaw 614 attached to the mounting plate 612. This configuration prevents the tissue manipulator from springing back toward its original configuration and also provides a second pinching point between the mounting plate and the mounting clamp that pinches the tissue manipulator in place between the mounting plate and the mounting clamp.

Step 1332 can be accomplished using any suitable technique or method of trimming a tissue manipulator and selection of a suitable technique or method can be based on various considerations, such as the material(s) that forms the tissue manipulator. Examples of suitable techniques and methods of trimming a tissue manipulator include cutting, using cutting tools (e.g., snips), grinding, and any other suitable technique or method considered suitable for a particular embodiment. FIG. 49 illustrates a trimmed tissue manipulator 808. For example, it is considered advantageous to trim the tissue manipulator such that about 10 millimeters from the location of the original placement of the shaft bend, or any other location, such that the new shaft end is disposed between, or adjacent to, the mounting clamp and mounting plate after assembly.

Step 1334 can be accomplished by providing instructions to a patient to phonate.

Step 1336 can be accomplished using any suitable device and/or method, such as by suturing the opening created in step 1302. Optionally, steps step 1302 can be repeated and another optional step comprising adjusting the tissue manipulator can be accomplished. Another optional step comprises removing a first tissue manipulator from a medical device and introducing a second tissue manipulator into the medical device and the arytenoid muscle.

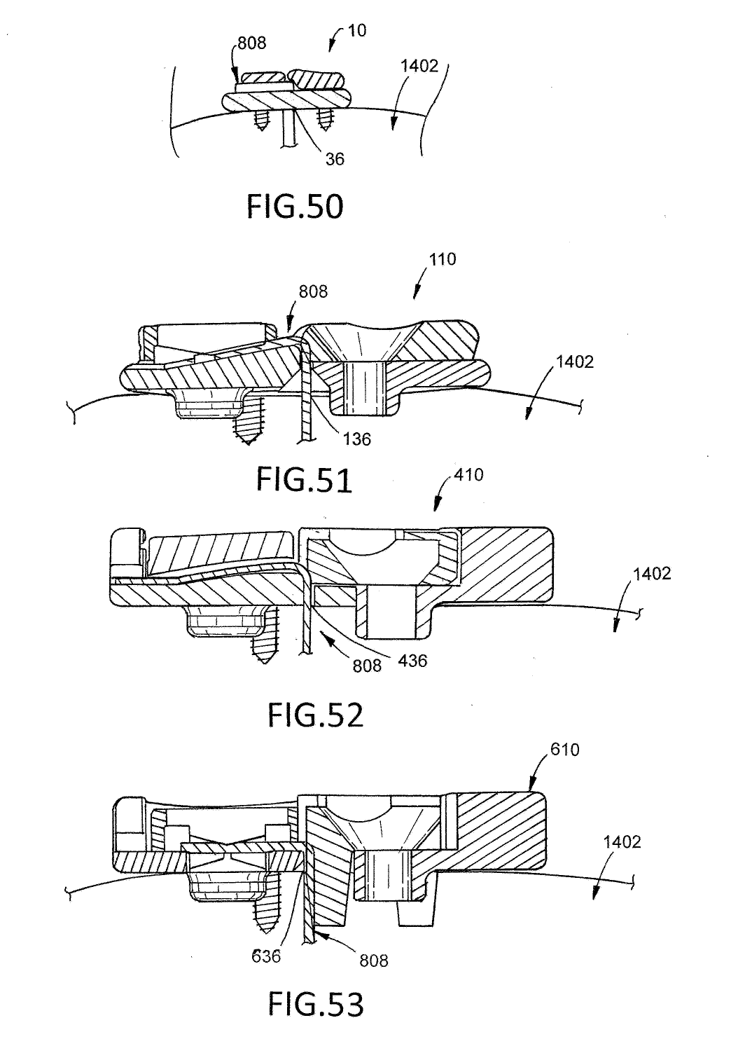

FIG. 50 illustrates the medical device 10 illustrated in FIG. 1 attached to the thyroid cartilage 1402 and the tissue manipulator 808 disposed through the mounting plate passageway 36 and the thyroid cartilage 1402. FIG. 51 illustrates the medical device 110 illustrated in FIG. 6 attached to the thyroid cartilage 1402 and the tissue manipulator 808 disposed through the mounting plate passageway 136 and the thyroid cartilage 1402. FIG. 52 illustrates the medical device 410 illustrated in FIG. 17 attached to the thyroid cartilage 1402 and the tissue manipulator 808 disposed through the mounting plate passageway 436 and the thyroid cartilage 1402. FIG. 53 illustrates the medical device 610 illustrated in FIG. 24 attached to the thyroid cartilage 1402 and a tissue manipulator 808 disposed through the mounting plate passageway 636 and the thyroid cartilage 1402.

While method 1300 has been described with respect to manipulating the position of an arytenoid muscle, any of the steps, alternative steps, and/or optional steps described herein can be utilized to treat any suitable tissue and a tissue fixation device can be attached to any suitable wall or feature of a patient. Selection of a suitable point of treatment to implant a tissue fixation device and a tissue manipulator according to an embodiment can be based on various considerations, including the treatment intended to be performed and the structural arrangement at the treatment site.

Those with ordinary skill in the art will appreciate that various modifications and alternatives for the described and illustrated embodiments can be developed in light of the overall teachings of the disclosure. Accordingly, the particular arrangements disclosed are intended to be illustrative only and not limiting as to the scope of the invention, which is to be given the full breadth of the appended claims and any and all equivalents thereof.

What is claimed is:

1. A tissue fixation system comprising:
a mounting plate having a mounting plate front surface, a curved mounting plate back surface opposably facing the mounting plate front surface, a mounting plate length measured along the mounting plate front surface, a mounting plate width measured along the mounting plate front surface and orthogonal to the mounting plate length, a mounting plate thickness extending from the mounting plate front surface to the mounting plate back surface, and a mounting plate main body defining a mounting plate passageway extending through the mounting plate thickness, a mounting plate track extending along a portion of the mounting plate width, and first, second, and third mounting plate apertures extending through the mounting plate thickness;
a mounting jaw releasably attached to the mounting plate and partially covering the mounting plate passageway, the mounting jaw having a mounting jaw main body defining a mounting jaw aperture;
a mounting clamp releasably attached to the mounting plate and partially disposed over the mounting plate track, the mounting clamp having a mounting clamp main body defining a mounting clamp first aperture and a mounting plate second aperture;
a plurality of attachment members, a first attachment member of the plurality of attachment members partially disposed through the mounting plate first aperture and the mounting jaw aperture and providing releasable attachment between the mounting plate and mounting jaw, a second attachment member of the plurality of attachment members partially disposed through the mounting plate second aperture and the mounting clamp first aperture and providing releasable attachment between the mounting plate and mounting clamp, and a third attachment member of the plurality of attachment members partially disposed through the mounting plate third aperture and the mounting clamp second aperture and providing releasable attachment between the mounting plate and mounting clamp; and
a tissue manipulator having a shaft and a coil, the shaft partially disposed within the mounting plate track and between the mounting plate and the mounting clamp, the shaft partially disposed between the mounting plate and the mounting jaw.

2. The tissue fixation system of claim 1, wherein the mounting plate main body defines a mounting plate recess that extends from the mounting plate front surface toward the mounting plate back surface to a mounting plate recess base;
wherein the mounting plate passageway extends from the mounting plate recess base to the mounting plate back surface;
wherein each of the mounting plate first aperture and mounting plate second aperture extends from the mounting plate recess base to the mounting plate back surface;
wherein the mounting plate track is disposed on the mounting plate recess base; and
wherein each of the mounting jaw and the mounting clamp are disposed within the mounting plate recess.

3. The tissue fixation system of claim 1, wherein the mounting plate track comprises a mounting plate track projection extending from the mounting plate front surface and away from the mounting plate back surface.

4. The tissue fixation system of claim 3, wherein the mounting plate track further comprises a mounting plate track recess extending into the mounting plate track and toward the mounting plate back surface.

5. The tissue fixation system of claim 3, wherein the mounting plate track has a mounting plate track first end, a mounting plate track second end, a mounting plate track projection first height at the mounting plate track first end, and a mounting plate track projection second height at the mounting plate track second end, the mounting plate track projection first height being greater than the mounting plate track projection second height.

6. The tissue fixation system of claim 1, wherein a portion of the mounting plate first aperture and a portion of the mounting plate second aperture are disposed on a first hypothetical plane, a portion of the mounting plate first aperture and a portion of the mounting plate third aperture are disposed on a second hypothetical plane, and a portion of the mounting plate first aperture, a portion of the mounting plate passageway, and a portion of the mounting plate track are disposed on a third hypothetical plane that is disposed between the first hypothetical plane and the second hypothetical plane.

7. The tissue fixation system of claim 6, wherein the third hypothetical plane is disposed at an angle to the first hypothetical plane that is less than 90 degrees.

8. The tissue fixation system of claim 6, wherein the third hypothetical plane is disposed at an angle to the second hypothetical plane that is less than 90 degrees.

9. The tissue fixation system of claim 1, wherein the mounting plate length is greater than the mounting plate width.

10. The tissue fixation system of claim 1, wherein the mounting clamp has a mounting clamp front surface and a mounting clamp back surface; and
wherein the mounting clamp defines a mounting clamp recess extending into the mounting clamp main body from the mounting clamp back surface toward the mounting clamp front surface, the mounting clamp having a mounting clamp first thickness within the recess, a mounting clamp second thickness within the recess, and a mounting clamp third thickness recess within the recess and disposed between the mounting clamp first thickness and mounting clamp second thickness, the mounting clamp third thickness being greater than the mounting clamp first thickness and the mounting clamp second thickness.

11. The tissue fixation system of claim 1, wherein the mounting plate main body defines fourth and fifth apertures extending through the mounting plate thickness.

12. The tissue fixation system of claim 1, wherein the mounting plate thickness is constant along the mounting plate length and mounting plate width.

13. A tissue fixation system comprising:
a mounting plate having a mounting plate front surface, a curved mounting plate back surface opposably facing the mounting plate front surface, a mounting plate length measured along the mounting plate front surface, a mounting plate width measured along the mounting plate front surface and orthogonal to the mounting plate length, a mounting plate thickness extending from the mounting plate front surface to the mounting plate back surface, and a mounting plate main body defining a mounting plate recess extending from the mounting plate front surface toward the mounting plate back surface to a mounting plate recess base, a mounting plate passageway extending from the mounting plate recess base to the mounting plate back surface, a mounting plate track disposed on the mounting plate recess base and extending along a portion of the mounting plate width, and first, second, third, fourth, and fifth mounting plate apertures extending through the mounting plate thickness, each of the mounting plate first aperture, mounting plate second aperture, and mounting plate third aperture extending from the mounting plate recess base to the mounting plate back surface, the mounting plate track comprising a mounting plate track projection extending from the mounting plate recess base and away from the mounting plate back surface;
a mounting jaw releasably attached to the mounting plate and partially covering the mounting plate passageway, the mounting jaw disposed within the mounting plate recess and having a mounting jaw main body defining a mounting jaw aperture;
a mounting clamp releasably attached to the mounting plate and partially disposed over the mounting plate track, the mounting clamp disposed within the mounting plate recess and having a mounting clamp main body defining a mounting clamp first aperture and a mounting plate second aperture;
a plurality of attachment members, a first attachment member of the plurality of attachment members partially disposed through the mounting plate first aperture and the mounting jaw aperture and providing releasable attachment between the mounting plate and mounting jaw, a second attachment member of the plurality of attachment members partially disposed through the mounting plate second aperture and the mounting clamp first aperture and providing releasable attachment between the mounting plate and mounting clamp, and a third attachment member of the plurality of attachment members partially disposed through the mounting plate third aperture and the mounting clamp second aperture and providing releasable attachment between the mounting plate and mounting clamp; and
a tissue manipulator having a shaft and a coil, the shaft partially disposed within the mounting plate track and between the mounting plate and the mounting clamp, the shaft partially disposed between the mounting plate and the mounting jaw.

14. The tissue fixation system of claim 13, wherein the mounting plate track further comprises a mounting plate track recess extending into the mounting plate track and toward the mounting plate back surface.

15. The tissue fixation system of claim 14, wherein the mounting plate track has a mounting plate track first end, a mounting plate track second end, a mounting plate track projection first height at the mounting plate track first end, and a mounting plate track projection second height at the mounting plate track second end, the mounting plate track projection first height being greater than the mounting plate track projection second height.

16. The tissue fixation system of claim 13, wherein a portion of the mounting plate first aperture and a portion of the mounting plate second aperture are disposed on a first hypothetical plane, a portion of the mounting plate first aperture and a portion of the mounting plate third aperture are disposed on a second hypothetical plane, and a portion of the mounting plate first aperture, a portion of the mounting plate passageway, and a portion of the mounting plate track are disposed on a third hypothetical plane that is disposed between the first hypothetical plane and the second hypothetical plane.

17. The tissue fixation system of claim 16, wherein the third hypothetical plane is disposed at an angle to the first hypothetical plane that is less than 90 degrees.

18. The tissue fixation system of claim 16, wherein the third hypothetical plane is disposed at an angle to the second hypothetical plane that is less than 90 degrees.

19. The tissue fixation system of claim 13, wherein the mounting clamp has a mounting clamp front surface and a mounting clamp back surface; and
wherein the mounting clamp defines a mounting clamp recess extending into the mounting clamp main body from the mounting clamp back surface toward the mounting clamp front surface, the mounting clamp having a mounting clamp first thickness within the recess, a mounting clamp second thickness within the recess, and a mounting clamp third thickness recess within the recess and disposed between the mounting clamp first thickness and mounting clamp second thickness, the mounting clamp third thickness being greater than the mounting clamp first thickness and the mounting clamp second thickness.

20. A tissue fixation system comprising:
a mounting plate having a mounting plate front surface, a curved mounting plate back surface opposably facing the mounting plate front surface, a mounting plate length measured along the mounting plate front surface, a mounting plate width measured along the mounting plate front surface and orthogonal to the mounting plate length, a mounting plate thickness extending from the mounting plate front surface to the mounting plate back surface, and a mounting plate main body defining a mounting plate recess extending from the mounting plate front surface toward the mounting plate back surface to a mounting plate recess base, a mounting plate passageway extending from the mounting plate recess base to the mounting plate back surface, a mounting plate track disposed on the mounting plate recess base and extending along a portion of the mounting plate width, and first, second, third, fourth, and fifth mounting plate apertures extending through the mounting plate thickness, each of the mounting plate first aperture, mounting plate second aperture, and mounting plate third aperture extending from the mounting plate recess base to the mounting plate back surface, the mounting plate track comprising a mounting plate track projection and a mounting plate track recess, the mounting plate projection extending from the mounting plate recess base and away from the mounting plate back surface, the mounting plate track recess extending into the mounting plate track and toward the mounting plate back surface;
a mounting jaw releasably attached to the mounting plate and partially covering the mounting plate passageway, the mounting jaw disposed within the mounting plate recess and having a mounting jaw main body defining a mounting jaw aperture;
a mounting clamp releasably attached to the mounting plate and partially disposed over the mounting plate track, the mounting clamp disposed within the mounting plate recess and having a mounting clamp main body defining a mounting clamp first aperture and a mounting plate second aperture;
a plurality of attachment members, a first attachment member of the plurality of attachment members partially disposed through the mounting plate first aperture and the mounting jaw aperture and providing releasable attachment between the mounting plate and mounting jaw, a second attachment member of the plurality of attachment members partially disposed through the mounting plate second aperture and the mounting clamp first aperture and providing releasable attachment between the mounting plate and mounting clamp, and a third attachment member of the plurality of attachment members partially disposed through the mounting plate third aperture and the mounting clamp second aperture and providing releasable attachment between the mounting plate and mounting clamp; and
a tissue manipulator having a shaft and a coil, the shaft partially disposed within the mounting plate track and between the mounting plate and the mounting clamp, the shaft partially disposed between the mounting plate and the mounting jaw;
wherein a portion of the mounting plate first aperture and a portion of the mounting plate second aperture are disposed on a first hypothetical plane, a portion of the mounting plate first aperture and a portion of the mounting plate third aperture are disposed on a second hypothetical plane, and a portion of the mounting plate first aperture, a portion of the mounting plate passageway, and a portion of the mounting plate track are disposed on a third hypothetical plane that is disposed between the first hypothetical plane and the second hypothetical plane;
wherein the third hypothetical plane is disposed at an angle to the first hypothetical plane that is less than 90 degrees; and
wherein the third hypothetical plane is disposed at an angle to the second hypothetical plane that is less than 90 degrees.
